(19) United States
(12) Patent Application Publication
(10) Pub. No.: US 2018/0369195 A1
(43) Pub. Date: Dec. 27, 2018

(54) COMBINATION THERAPY FOR TREATMENT OF MELANOMA

(71) Applicant: The Regents of the University of California, Oakland, CA (US)

(72) Inventors: Gary L. Firestone, Oakland, CA (US); Aishwarya Kundu, Fremont, CA (US)

(21) Appl. No.: 15/778,601

(22) PCT Filed: Nov. 29, 2016
(86) PCT No.: PCT/US16/64037

§ 371 Date: May 23, 2018

Related U.S. Application Data
(60) Provisional application No. 62/261,020, filed on Nov. 30, 2015.

(51) Int. Cl. A61K 31/404; A61P 35/00

(52) U.S. Cl. CPC A61K 31/404; A61K 31/437; A61K 2300/00; A61P 35/00

(57) ABSTRACT

The present disclosure provides a pharmaceutical composition that inhibits the proliferation of a cancer cell, comprising a first compound selected from indole-3-carbinol (I3C), 1-benzyl I3C, or a pharmaceutically acceptable salt or ester thereof, and a second compound that binds to and inhibits: i) an oncogenic RAF polypeptide at a site that is distinct from the site at which the first compound binds; ii) a polypeptide of the RAF signaling pathway; iii) a polypeptide of the Wnt-β-catenin signaling pathway; iv) or a polypeptide of the PI3K/AKT/mTOR signaling pathway. The present disclosure also provides a method of treating cancer in a subject.

### CROSS-REFERENCE

This application claims the benefit of U.S. Provisional Patent Application No. 62/261,020, filed Nov. 30, 2015, which application is incorporated herein by reference in its entirety.

### STATEMENT REGARDING FEDERALLY SPONSORED RESEARCH

This invention was made with Government support under contracts CA102360 and CA164095 awarded by the National Cancer Institute. The Government has certain rights in the invention.

### INTRODUCTION

Human melanoma, the most aggressive form of malignant skin cancer, can be categorized by distinct mutational profiles that determine their corresponding cellular phenotypes, proliferative capabilities and therapeutic options. Well-established driver mutations of melanoma include expression of oncogenic forms of BRAF and NRAS and loss of tumor suppressor proteins such as PTEN, TP53 and p16INK4a. Notably, in more than 60% of melanoma patients, a point mutation occurs within the BRAF gene, with approximately 90% of these mutations being T1799A that substitutes Valine for Glutamate at residue 600 in the kinase domain forming the constitutively active Ser/Thr protein kinase BRAF-V600E. In human melanomas, a key downstream target of oncogenic BRAF signaling, which triggers constitutive hyperactivation of MEK and Erk/MAPK, is the enhanced expression of Microphthalmia Associated Transcription Factor isoform M (MITF-M), the master regulator of melanocyte and melanoma biology. MITF-M is a ‘lineage survival’ oncogene that is required for both tissue-specific tumorigenesis and progression and elevated MITF-M levels correlate with decreased overall patient survival. Furthermore, one mechanism of acquired resistance to BRAF inhibitors involves the amplification and restoration of transcriptional activity of MITF-M.

The incidence of skin cancer over the past three decades have been more than all other cancers combined. It is estimated that one in five Americans will develop skin cancer in the course of their lifetime. Melanoma, a malignant form of skin cancer originating in the melanin containing melanocytes, accounts for less than 2 percent of all diagnosed skin cancer cases, but causes 77% of all skin cancer deaths. Death resulting from melanoma has an alarming rate of one every 57 minutes. The overall 5-year survival rate for patients is dependent on the stage of detection and outcome is dismal if diagnosis happens after the disease has metastasized to distant organs.

There is a need in the art for methods of treating melanoma.

### SUMMARY

The present disclosure provides a pharmaceutical composition that inhibits the proliferation of a cancer cell, comprising a first compound selected from indole-3-carbinol (I3C), 1-benzyl I3C, or a pharmaceutically acceptable salt or ester thereof, and a second compound that binds to and inhibits: i) an oncogenic RAF polypeptide at a site that is distinct from the site at which the first compound binds; ii) a polypeptide of the RAF signaling pathway; iii) a polypeptide of the Wnt-β-catenin signaling pathway; iv) or a polypeptide of the PI3K/AKT/mTOR signaling pathway. The present disclosure also provides a method of treating cancer in a subject, the method comprising administering to the subject an effective amount of a first compound selected from I3C, 1-benzyl I3C, or a pharmaceutically acceptable salt or ester thereof, and a second compound that binds to and inhibits: i) an oncogenic RAF polypeptide at a site that is distinct from the site at which the first compound binds; ii) a polypeptide of the RAF signaling pathway; iii) a polypeptide of the Wnt-β-catenin signaling pathway; iv) or a polypeptide of the PI3K/AKT/mTOR signaling pathway

The present disclosure provides a pharmaceutical composition that inhibits the proliferation of a cancer cell (e.g., melanoma). The pharmaceutical composition comprises a first and second compound, wherein the second compound binds an oncogenic RAF polypeptide (e.g., BRAF-V600E polypeptide). In some cases, the composition comprises a second compound that binds at or near the ATP pocket of the oncogenic RAF polypeptide. In some cases, the second compound binds at or near the ATP pocket of BRAF-V600E (e.g., Vemurafenib).

The present disclosure also provides a method of treating cancer in a subject, comprising administering to the subject an effective amount of a composition comprising a first compound selected from indole-3-carbinol (I3C), 1-benzyl I3C, or a pharmaceutically acceptable salt or ester thereof, and a second compound that binds an oncogenic RAF polypeptide at a site that is distinct from the site at which the first compound binds.

The present disclosure provides a method of treating cancer in a subject, comprising administering to the subject an effective amount of a composition comprising a first and second compound, wherein the second compound binds an oncogenic RAF polypeptide (e.g., BRAF-V600E polypeptide). In some cases, the composition comprises a second compound that binds at or near the ATP pocket of the oncogenic RAF polypeptide. In some cases, the second compound binds at or near the ATP pocket of BRAF-V600E (e.g., Vemurafenib).

The present disclosure provides a method of treating cancer in a subject, comprising administering to the subject an effective amount of a composition comprising a first and second compound, wherein the second compound inhibits a RAF signaling pathway polypeptide. For example, the compound inhibits one of c-Kit, RAS, MIK, ERK, and BRN-2. In some cases, the second compound binds a Wnt-β-catenin signaling pathway polypeptide. In some cases, the second compound binds a PI3K/AKT/mTOR signaling pathway polypeptide. A composition of the present disclosure is in some cases administered orally, and in other cases, the composition is administered topically.

The present disclosure provides a pharmaceutical composition that inhibits proliferation of a cancer cell, comprising: a) a first compound selected from indole-3-carbinol, 1-benzyl indole-3-carbinol, or a pharmaceutically acceptable salt or ester thereof; and b) a second compound that binds to and inhibits a polypeptide selected from: i. an oncogenic RAF polypeptide, wherein the second compound binds at a site that is distinct from the site at which the first compound binds; ii. a polypeptide of the RAF signaling pathway selected from c-KIT, RAS, MEK, ERK, or BRN-2; iii. a polypeptide of the Wnt-β-catenin signaling pathway; or iv. a polypeptide of the PI3K/AKT/mTOR signaling pathway. In some cases, the second compound binds to and inhibits an oncogenic RAF polypeptide. In some cases, the second compound binds at or near the ATP pocket of the oncogenic RAF polypeptide. In some cases, the second compound is Vemurafenib. In some cases, the second compound is Dabrafenib. In some cases, the cancer is melanoma. In some cases, the cancer is BRAF-inhibitor-resistant melanoma. In some cases, the cancer expresses an oncogenic mutation in BRAF. In some cases, the cancer is colon, thyroid or lung cancer. In some cases, the oncogenic BRAF polypeptide comprises a BRAF-V600 mutation. In some cases, the BRAF-V600 mutation is V600E, V600K, V600D or V600R.

The present disclosure provides a method of treating cancer in a subject, the method comprising administering to the subject combined effective amounts of: a) a first compound selected from indole-3-carbinol, 1-benzyl indole-3-carbinol, or a pharmaceutically acceptable salt or ester thereof; and b) a second compound that binds to and inhibits a polypeptide selected from: i) an oncogenic BRAF polypeptide, where the second compound binds at a site that is distinct from the site at which the first compound binds; ii) a polypeptide of the RAF signaling pathway selected from c-KIT, RAS, MEK, ERK, or BRN-2; iii) a polypeptide of the Wnt-β-catenin signaling pathway; and v) a polypeptide of the PI3K/AKT/mTOR signaling pathway. In some cases, the second compound is Vemurafenib. In some cases, the second compound is Dabrafenib. In some cases, the cancer is melanoma. In some cases, the cancer is BRAF-inhibitor-resistant melanoma. In some cases, the cancer expresses an oncogenic mutation in BRAF. In some cases, the cancer is colon, thyroid or lung cancer. In some cases, the first and the second compounds are present in a pharmaceutical composition, either in separate formulations or in the same formulation. In some cases, the first compound is administered orally. In some cases, the second compound is administered orally. In some cases, the first compound is administered topically. In some cases, the second compound is administered topically. In some cases, the first compound is administered intravenously. In some cases, the second compound is administered intravenously. In some cases, the first compound is administered intramuscularly. In some cases, the second compound is administered intramuscularly. In some cases, the first compound and the second compound are administered in the same formulation. In some cases, the first compound and the second compound are administered in separate formulations. In some cases, the first compound and the second compound are administered substantially simultaneously. In some cases, the first compound and the second compound are administered within 1 hour to 24 hours of one another. In some cases, the first compound is 1-benzyl-I3C and the second compound is Vemurafenib. In some cases, the 1-benzyl-I3C is administered in an amount of from about 1 mg/kg to about 50 mg/kg. In some cases, the Vemurafenib is administered in an amount of from 100 mg/kg to about 500 mg/kg. In some cases, the individual is a human.

### BRIEF DESCRIPTION OF THE DRAWINGS

FIG. 1A-1D depict the various effects of indole-3-carbinol (I3C).

FIG. 2 depicts the dose dependent cell cycle effects of I3C.

FIG. 3A-3B depict the cell cycle effects of I3C in melanoma cells.

FIG. 4A-4F depicts the effects of I3C on MITF-M signaling.

FIG. 5A-5C depicts the effects of I3C on BRAF signaling.

FIG. 6A-6E depict the effect of I3C on BRAF-V600E.

FIG. 7A-7D depict the combinatorial effects of I3C and Vemurafenib.

FIG. 8A-8C depict the various effects of 1-benzyl I3C.

FIG. 9A-9G depict dose dependent and temporal effects of 1-benzyl I3C.

FIG. 10A-10D depict regulation of MITF-M expression by 1-benzyl I3C.

FIG. 11A-11B depict the effect of 1-benzyl I3C on Wnt signaling.

FIG. 12A-12C depict canonical Wnt signaling in 1-benzyl I3C's function.

FIG. 13A-13C depict canonical Wnt signaling in 1-benzyl I3C's function.

FIG. 14A-14B depict the combinatorial effects of 1-benzyl I3C and Vemurafenib.

### DEFINITIONS

As used herein, a “pharmaceutical composition” is meant to encompass a composition suitable for administration to a subject, such as a mammal, especially a human. In general a “pharmaceutical composition” is sterile, and is free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal, inhalational, and the like. In some embodiments a subject composition is formulated with an excipient other than dimethylsulfoxide (DMSO). In other embodiments, the pharmaceutical compositions are suitable for administration by a route other than transdermal administration. A pharmaceutical composition will in some embodiments include a subject compound and a pharmaceutically acceptable excipient. In some embodiments, a pharmaceutically acceptable excipient is other than DMSO.

The terms “compound”, “agent” and “active agent”, are used interchangeably herein.

Any conventional carrier or excipient may be used in the pharmaceutical compositions of the embodiments. The choice of a particular carrier or excipient, or combinations of carriers or excipients, will depend on the mode of administration being used to treat a particular patient or type of medical condition or disease state. In this regard, the preparation of a suitable pharmaceutical composition for a particular mode of administration is well within the scope of those skilled in the pharmaceutical arts. Additionally, the ingredients for such compositions are commercially available from, for example, Sigma, P.O. Box 14508, St. Louis, Mo. 63178. By way of further illustration, conventional formulation techniques are described in Remington: The Science and Practice of Pharmacy, 20th Edition, Lippincott Williams & White, Baltimore, Md. (2000); and H. C. Ansel et al., Pharmaceutical Dosage Forms and Drug Delivery Systems, 7th Edition, Lippincott Williams & White, Baltimore, Md. (1999).

Representative examples of materials which can serve as pharmaceutically acceptable carriers or excipients include, but are not limited to, the following: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) compressed propellant gases, such as chlorofluorocarbons and hydrofluorocarbons; and (22) other non-toxic compatible substances employed in pharmaceutical compositions.

The term “pharmaceutically acceptable salt” means a salt which is acceptable for administration to a patient, such as a mammal (e.g., a human, e.g., a human with melanoma) (salts with counter ions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

As used herein, “pharmaceutically acceptable derivatives” of a compound of the invention include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and are either pharmaceutically active or are prodrugs.

“Pharmaceutically effective amount” and “therapeutically effective amount” refer to the amount of a compound that, when administered to a mammal or other subject for treating a disease, is sufficient to effect such treatment for the disease. The “therapeutically effective amount” will vary depending on the compound or the cell, the disease and its severity and the age, weight, etc., of the subject to be treated.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc. In some cases, the individual is a human.

The term “treating” or “treatment” as used herein means the treating or treatment of a disease or medical condition in a patient, such as a mammal (e.g., a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient.

The term “synergy” or “synergistic effect” as used herein means that a combination of agents provides for an effect greater than any of each single agent, which effect may be greater than the additive effect of the combined agents.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a method of treatment” includes a plurality of such method of treatment and reference to “the pharmaceutical composition” includes reference to one or more pharmaceutical compositions and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a pharmaceutical composition that inhibits proliferation of a cancer cell (e.g., melanoma cell), comprising a first and second compound. The first compound of a composition of the present disclosure is indole-3-carbinol (I3C), 1-benzyl I3C, or a pharmaceutically acceptable salt or ester thereof. In some cases, the second compound of a composition of the present disclosure binds an oncogenic RAF polypeptide (e.g., an oncogenic BRAF polypeptide (e.g., BRAF-V600E polypeptide); an oncogenic A-RAF polypeptide; an oncogenic C-RAF polypeptide) at a site that is distinct from the site at which the first compound binds. In some cases, the second compound of a composition of the present disclosure binds and inhibits a polypeptide of the RAF signaling pathway (e.g., the compound inhibits c-Kit, RAS, MEK, ERK, or BRN-2). In some cases, the second compound of a composition of the present disclosure binds to and inhibits a polypeptide of the Wnt-β-catenin signaling pathway. In some cases, the second compound of a composition of the present disclosure binds to and inhibits a polypeptide of the PI3K/AKT/mTOR signaling pathway. Also provided are methods of treating cancer (e.g., melanoma) in a subject, methods comprising administering to the subject an effective amount of a composition of the present disclosure.

### Pharmaceutical Compositions

The present disclosure provides a pharmaceutical composition that inhibits proliferation of a cancer cell (e.g., melanoma cell), comprising a first and second compound. The first compound of a composition of the present disclosure is indole-3-carbinol (I3C), 1-benzyl I3C, or a pharmaceutically acceptable salt or ester thereof. In some cases, the second compound of a composition of the present disclosure binds an oncogenic RAF polypeptide (e.g., an oncogenic BRAF polypeptide (e.g., BRAF-V600E polypeptide); an oncogenic A-RAF polypeptide; an oncogenic C-RAF polypeptide) at a site that is distinct from the site at which the first compound binds. In some cases, the second compound of a composition of the present disclosure binds and inhibits a polypeptide of the RAF signaling pathway (e.g., the compound inhibits c-Kit, RAS, MEK, ERK, or BRN-2). In some cases, the second compound of a composition of the present disclosure binds to and inhibits a polypeptide of the Wnt-β-catenin signaling pathway. In some cases, the second compound of a composition of the present disclosure binds to and inhibits a polypeptide of the PI3K/AKT/mTOR signaling pathway.

### Indole Compounds

In some cases, a composition of the present disclosure that inhibits proliferation of a cancer cell comprises indole-3-carbinol (I3C). In some cases, I3C that is used in a composition of the present disclosure binds the catalytic loop of an oncogenic BRAF polypeptide. In some cases, I3C binds the His-Arg-Asp (HRD) motif of the catalytic loop of an oncogenic BRAF polypeptide (e.g., BRAF-V600E). In some cases, I3C binds an oncogenic BRAF polypeptide via Van der Waals interactions with two critical catalytic residues Arg 575 and Asp 576 that are located within the HRD motif. I3C of use in the present disclosure can form Van der Waals interactions with Met 620, Val 624, Ser 616, Tyr 633, Leu 577, Ala 641, Tyr 619, and Lys 578 that are generally within the catalytic loop.

I3C that is of use in a composition of the present disclosure has the formula:

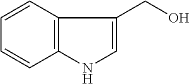 [SMILES: OCc1c[nH]c2ccccc12]

In some cases, indole-3-carbinol (I3C) can inhibit proliferation of a cancer cell with an EC₅₀ (half maximal effective concentration) of from about 1 nM to about 1 mM, e.g., from about 1 nM to about 10 nM, from about 10 nM to about 15 nM, from about 15 nM to about 25 nM, from about 25 nM to about 50 nM, from about 50 nM to about 75 nM, from about 75 nM to about 100 nM, from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 450 nM, from about 450 nM to about 500 nM, from about 500 nM to about 750 nM, from about 750 nM to about 1 μM, from about 1 μM to about 10 μM, from about 10 μM to about 25 μM, from about 25 μM to about 50 μM, from about 50 μM to about 75 μM, from about 75 μM to about 100 μM, from about 100 μM to about 250 μM from about 250 μM to about 500 μM, or from about 500 μM to about 1 mM.

In some cases, a composition of the present disclosure that inhibits proliferation of a cancer cell comprises a derivative of indole-3-carbinol (I3C). Examples of I3C derivatives can be found in, e.g., U.S. Pat. Nos. 6,001,868; 6,150,395; 6,369,095 and 6,656,963, herein incorporated by reference. In some cases, the I3C derivative that is used in a composition of the present disclosure binds the catalytic loop of an oncogenic BRAF polypeptide. In some cases, the I3C derivative binds the His-Arg-Asp (HRD) motif of the catalytic loop of an oncogenic BRAF polypeptide (e.g., BRAF-V600E). An I3C derivative of the present disclosure can form Van der Waals interactions with Met 620, Val 624, Ser 616, Tyr 633, Leu 577, Ala 641, Tyr 619, and Lys 578 that are generally within the catalytic loop. In some cases, the I3C derivative binds an oncogenic BRAF polypeptide via Van der Waals interactions with two critical catalytic residues Arg 575 and Asp 576 that are located within the HRD motif.

An I3C derivative that finds use in the present disclosure is a synthetic analog of I3C. A synthetic analog of I3C that inhibits proliferation of a cancer cell can exhibit enhanced potency in comparison to the potency of I3C. Accordingly, a synthetic analog of I3C can exhibit about 10-fold enhanced potency (e.g., 1-10 fold, 1-5 fold, 5-10 fold enhancement, etc.), about 100-fold enhanced potency (e.g., 10-50 fold, 50-100 fold, 20-80 fold enhancement, etc.), about 1000-fold enhanced potency (e.g., 100-500 fold, 500-1000 fold, 200-800 fold enhancement, etc.), more than 1000-fold enhanced potency in inhibiting proliferation of a cancer cell. An I3C derivative may not exhibit enhanced potency in inhibiting proliferation of a cancer cell in comparison to the potency of I3C (e.g., may exhibit decreased potency).

An I3C derivative that finds use in the present disclosure is 1-benzyl I3C and has the formula:

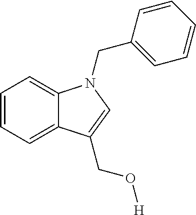 [SMILES: OCc1cn(Cc2ccccc2)c2ccccc12]

In some cases, 1-benzyl I3C can inhibit proliferation of a cancer cell with an EC₅₀ (half maximal effective concentration) of from about 1 nM to about 1 mM, e.g., from about 1 nM to about 10 nM, from about 10 nM to about 15 nM, from about 15 nM to about 25 nM, from about 25 nM to about 50 nM, from about 50 nM to about 75 nM, from about 75 nM to about 100 nM, from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 450 nM, from about 450 nM to about 500 nM, from about 500 nM to about 750 nM, from about 750 nM to about 1 μM, from about 1 μM to about 10 μM, from about 10 μM to about 25 μM, from about 25 μM to about 50 μM, from about 50 μM to about 75 μM, from about 75 μM to about 100 μM, from about 100 μM to about 250 μM from about 250 μM to about 500 μM, or from about 500 μM to about 1 mM.

The present disclosure provides a pharmaceutical composition that inhibits proliferation of a cancer cell (e.g., melanoma cell), comprising a first and second compound. The first compound of a composition of the present disclosure is indole-3-carbinol (I3C), 1-benzyl I3C, or a pharmaceutically acceptable salt or ester thereof. In some cases, a pharmaceutically acceptable salt or ester thereof can inhibit proliferation of a cancer cell with an EC₅₀ (half maximal effective concentration) of from about 1 nM to about 1 mM, e.g., from about 1 nM to about 10 nM, from about 10 nM to about 15 nM, from about 15 nM to about 25 nM, from about 25 nM to about 50 nM, from about 50 nM to about 75 nM, from about 75 nM to about 100 nM, from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 450 nM, from about 450 nM to about 500 nM, from about 500 nM to about 750 nM, from about 750 nM to about 1 μM, from about 1 μM to about 10 μM, from about 10 μM to about 25 μM, from about 25 μM to about 50 μM, from about 50 μM to about 75 μM, from about 75 μM to about 100 μM, from about 100 μM to about 250 μM from about 250 μM to about 500 μM, or from about 500 μM to about 1 mM.

The present disclosure provides a pharmaceutical composition that inhibits proliferation of a cancer cell (e.g., melanoma cell), comprising a first and second compound. The first compound of a composition of the present disclosure is indole-3-carbinol (I3C), 1-benzyl I3C, or a pharmaceutically acceptable salt or ester thereof. In some cases, the second compound of a composition of the present disclosure binds an oncogenic RAF polypeptide (e.g., an oncogenic BRAF polypeptide (e.g., BRAF-V600E polypeptide); an oncogenic A-RAF polypeptide; an oncogenic C-RAF polypeptide) at a site that is distinct from the site at which the first compound binds. In some cases, the second compound of a composition of the present disclosure binds and inhibits a polypeptide of the RAF signaling pathway (e.g., the compound inhibits c-Kit, RAS, MEK, ERK, or BRN-2). In some cases, the second compound of a composition of the present disclosure binds to and inhibits a polypeptide of the Wnt-β-catenin signaling pathway. In some cases, the second compound of a composition of the present disclosure binds to and inhibits a polypeptide of the PI3K/AKT/mTOR signaling pathway.

Compounds that Inhibit an Oncogenic RAF Polypeptide

In some cases, the second compound of a composition of the present disclosure is a compound that binds an oncogenic BRAF polypeptide (e.g., BRAF-V600E) at a site that is distinct from the site at which the first compound binds, i.e., a site that is distinct from the catalytic loop of an oncogenic BRAF polypeptide. In some cases, the second compound binds at or near the ATP pocket of the oncogenic BRAF polypeptide. For example, a second compound of a composition of the present disclosure can be Vemurafenib, which is a competitive inhibitor for the ATP binding site of an oncogenic BRAF polypeptide.

A second compound that finds use in a composition of the present disclosure is Vemurafenib and has the formula:

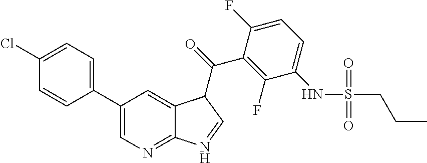 [SMILES: CCCS(=O)(=O)Nc1ccc(F)c(C(=O)C2C=Nc3ncc(-c4ccc(Cl)cc4)cc32)c1F]

In some cases, Vemurafenib can inhibit proliferation of a cancer cell with an EC₅₀ (half maximal effective concentration) of from about 1 nM to about 1 mM, e.g., from about 1 nM to about 10 nM, from about 10 nM to about 15 nM, from about 15 nM to about 25 nM, from about 25 nM to about 50 nM, from about 50 nM to about 75 nM, from about 75 nM to about 100 nM, from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 450 nM, from about 450 nM to about 500 nM, from about 500 nM to about 750 nM, from about 750 nM to about 1 μM, from about 1 μM to about 10 μM, from about 10 μM to about 25 μM, from about 25 μM to about 50 μM, from about 50 μM to about 75 μM, from about 75 μM to about 100 μM, from about 100 μM to about 250 μM from about 250 μM to about 500 μM, or from about 500 μM to about 1 mM.

A second compound that finds use in a composition of the present disclosure is Dabrafenib and has the formula:

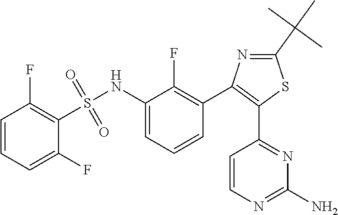 [SMILES: CC(C)(C)c1nc(-c2cccc(NS(=O)(=O)c3c(F)cccc3F)c2F)c(-c2ccnc(N)n2)s1]

In some cases, Dabrafenib can inhibit proliferation of a cancer cell with an EC₅₀ (half maximal effective concentration) of from about 1 nM to about 1 mM, e.g., from about 1 nM to about 10 nM, from about 10 nM to about 15 nM, from about 15 nM to about 25 nM, from about 25 nM to about 50 nM, from about 50 nM to about 75 nM, from about 75 nM to about 100 nM, from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 450 nM, from about 450 nM to about 500 nM, from about 500 nM to about 750 nM, from about 750 nM to about 1 μM, from about 1 μM to about 10 μM, from about 10 μM to about 25 μM, from about 25 μM to about 50 μM, from about 50 μM to about 75 μM, from about 75 μM to about 100 μM, from about 100 μM to about 250 μM from about 250 μM to about 500 μM, or from about 500 μM to about 1 mM.

Compounds that Inhibit a RAF Signaling Pathway Polypeptide

In some cases, the second compound of a composition of the present disclosure is a compound that inhibits a RAF signaling pathway polypeptide. For example, the compound inhibits one of c-Kit, RAS, MIK, ERK, and BRN-2.

Suitable MEK inhibitors include trametinib and cobimetinib. Suitable MEK inhibitors include binimetinib. Suitable MEK inhibitors include selumetinib. Suitable MEK inhibitors include refametinib.

Selumetinib is a compound of the formula:

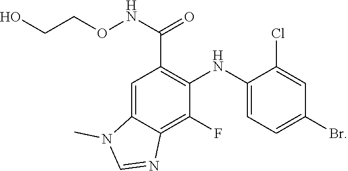 [SMILES: Cn1cnc2c(F)c(Nc3ccc(Br)cc3Cl)c(C(=O)NOCCO)cc21]

Refametinib is a compound of the formula:

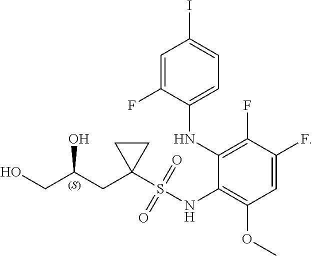

Binimetinib is a compound of the formula:

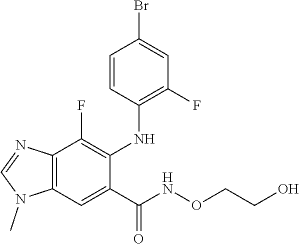 [SMILES: Cn1cnc2c(F)c(Nc3ccc(Br)cc3F)c(C(=O)NOCCO)cc21]

Trametinib is a compound of the formula:

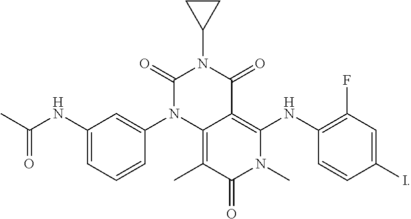 [SMILES: CC(=O)Nc1cccc(-n2c(=O)n(C3CC3)c(=O)c3c(Nc4ccc(I)cc4F)n(C)c(=O)c(C)c32)c1]

Cobimetinib is a compound of the formula:

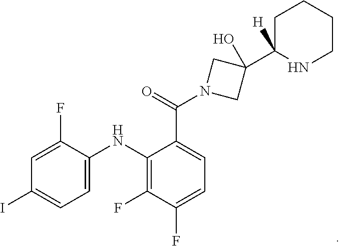 [SMILES: O=C(c1ccc(F)c(F)c1Nc1ccc(I)cc1F)N1CC(O)([C@@H]2CCCCN2)C1]

Suitable c-Kit inhibitors include matinib (Gleevec) and nilotinib (Tasigna).

Imatinib is a compound of the formula:

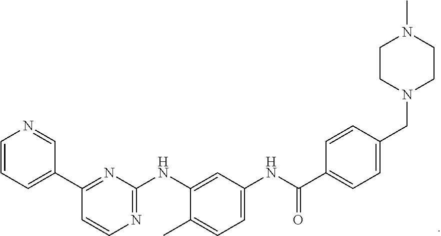 [SMILES: Cc1ccc(NC(=O)c2ccc(CN3CCN(C)CC3)cc2)cc1Nc1nccc(-c2cccnc2)n1]

Nilotinib is a compound of the formula:

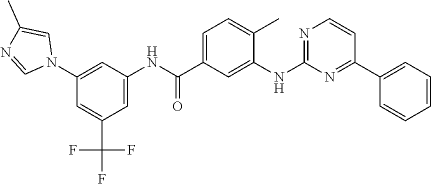 [SMILES: Cc1cn(-c2cc(NC(=O)c3ccc(C)c(Nc4nccc(-c5ccccc5)n4)c3)cc(C(F)(F)F)c2)cn1]

Suitable ERK inhibitors include a compound of the following structure:

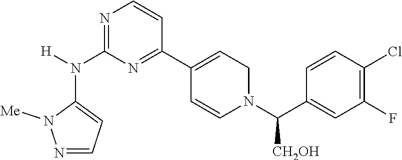 [SMILES: Cn1nccc1Nc1nccc(C2=CCN([C@H](CO)c3ccc(Cl)c(F)c3)C=C2)n1]

Suitable ERK inhibitors include a compound of the following structure:

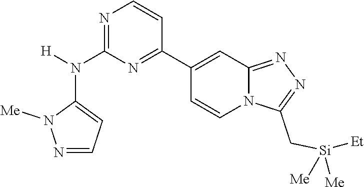 [SMILES: CC[Si](C)(C)Cc1nnc2cc(-c3ccnc(Nc4ccnn4C)n3)ccn12]

Suitable ERK inhibitors include a compound of the following structure:

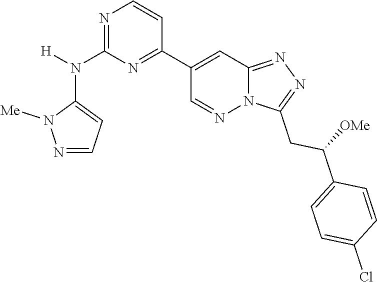 [SMILES: CO[C@@H](Cc1nnc2cc(-c3ccnc(Nc4ccnn4C)n3)cnn12)c1ccc(Cl)cc1]

Suitable ERK inhibitors include a compound of the following structure:

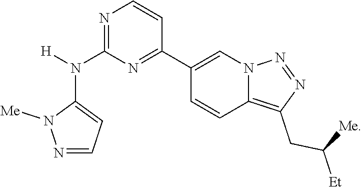 [SMILES: CC[C@H](C)Cc1nnn2cc(-c3ccnc(Nc4ccnn4C)n3)ccc12]

Suitable ERK inhibitors include ERK inhibitors described in WO 2012/094313, WO 2012/087772, and WO 2012/030685.

Compounds that Inhibit a Polypeptide of the Wnt-β-Catenin Pathway

Compounds that inhibit a polypeptide of the Wnt-β-catenin pathway include, e.g., OTSA101, OMP-54F28, Vantictumab, and Foxy-5. Other compounds that inhibit a polypeptide of the Wnt-β-catenin pathway include, e.g., compounds that target Porcupine (e.g., IWP, LGK974, C59).

Vantictumab is described in U.S. Pat. No. 8,507,442. Antibodies that target FZD10 are described in U.S. Pat. No. 9,139,655.

IWP is a compound of the formula:

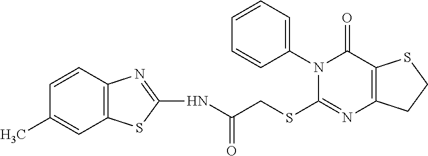 [SMILES: Cc1ccc2nc(NC(=O)CSc3nc4c(c(=O)n3-c3ccccc3)SCC4)sc2c1]

LGK974 is a compound of the formula:

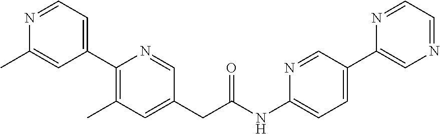 [SMILES: Cc1cc(-c2ncc(CC(=O)Nc3ccc(-c4cnccn4)cn3)cc2C)ccn1]

C59 is a compound of the formula:

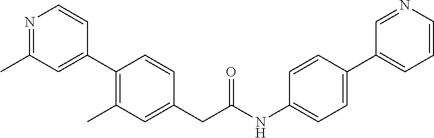 [SMILES: Cc1cc(-c2ccc(CC(=O)Nc3ccc(-c4cccnc4)cc3)cc2C)ccn1]

Compounds that inhibit a polypeptide of the Wnt-β-catenin pathway include, compounds that target tankyrase and/or Axin (e.g., XAV939, IWR-endo, G007-LK, G244-LM),

XAV939 is a compound of the formula:

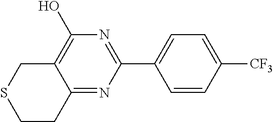 [SMILES: Oc1nc(-c2ccc(C(F)(F)F)cc2)nc2c1CSCC2]

IWR-endo is a compound of the formula:

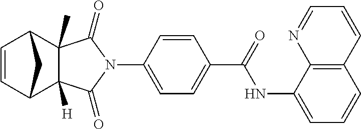 [SMILES: C[C@@]12C(=O)N(c3ccc(C(=O)Nc4cccc5cccnc45)cc3)C(=O)[C@@H]1[C@@H]1C=C[C@H]2C1]

G007-LK is a compound of the formula:

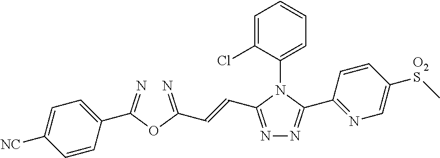 [SMILES: CS(=O)(=O)c1ccc(-c2nnc(/C=C/C(=N)OC(=N)c3ccc(C#N)cc3)n2-c2ccccc2Cl)nc1]

Compounds that inhibit a polypeptide of the Wnt-β-catenin pathway include, compounds that target TCF and/or β-catenin (e.g., 2,4-diamino-quinazoline, Quercetin, PKF115-584, BC2059).

2,4-diamino-quinazoline is a compound of the formula:

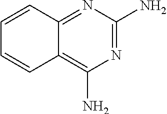 [SMILES: Nc1nc(N)c2ccccc2n1]

Quercetin is a compound comprising the formula:

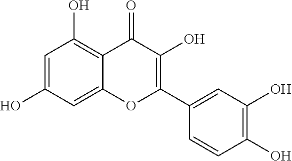 [SMILES: O=c1c(O)c(-c2ccc(O)c(O)c2)oc2cc(O)cc(O)c12]

Compounds that inhibit a polypeptide of the Wnt-β-catenin pathway are agents that antagonize signaling by Wnts, either by binding to Wnts or Wnt receptors and blocking Wnt-Wnt receptor interaction or by modulating intracellular signaling activity downstream of Wnt binding, e.g. by stabilizing β-catenin. Naturally occurring factors that inhibit Wnt signaling include, without limitation, the Dickkopf proteins (DKK-1 to -4), secreted Frizzled-related proteins (sFRP-1 to -5), Wnt Inhibitory Factor1 (WIF1), adenomatosis polyposis down-regulated 1 (APCDD1), and Soggy/DKKL1. Other inhibitors of Wnt signaling include Frizzled-Fc fusion proteins, e.g. Frizzled 8-Fc (Fz8-Fc), antibodies specific for Wnts or Wnt receptors, small molecule compounds such as pyrvinium, IWP2. Other compounds that inhibit a polypeptide of the Wnt-β-catenin pathway include those disclosed in U.S. patent application Ser. No. 10/678,639, hereby incorporated by reference.

Compounds that Inhibit a Polypeptide of the PI3K/AKT/mTOR Pathway

Compounds that inhibit a polypeptide of the PI3K/AKT/mTOR pathway include, e.g., compounds that target mTORC1 (e.g., Everolimus, Temsirolimus).

Everolimus is a compound of the formula:

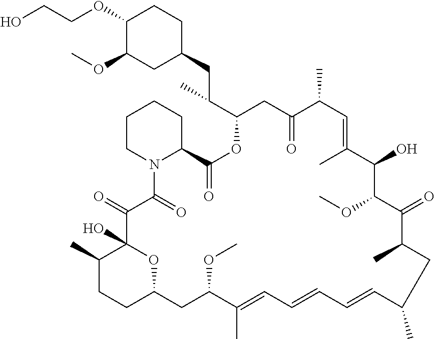 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

Temsirolimus is a compound of the formula:

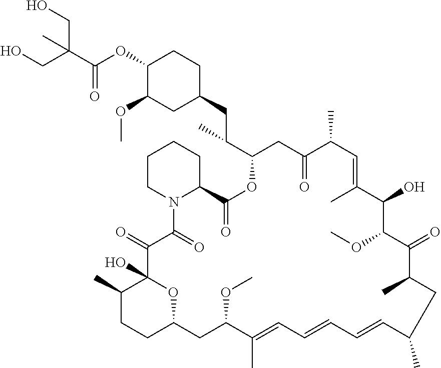 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C]

Compounds that inhibit a polypeptide of the PI3K/AKT/mTOR pathway include, e.g., compounds that target PI3K/mTOR (e.g., BEZ235, GDC-0980, PF-05212384, SAR245409).

BEZ235 is a compound of the formula:

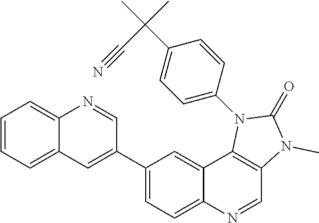 [SMILES: Cn1c(=O)n(-c2ccc(C(C)(C)C#N)cc2)c2c3cc(-c4cnc5ccccc5c4)ccc3ncc21]

GDC-0980 is a compound of the formula:

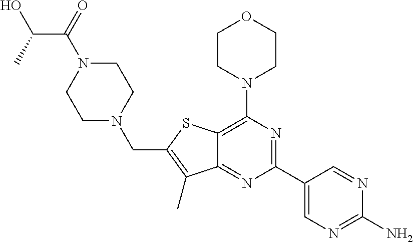 [SMILES: Cc1c(CN2CCN(C(=O)[C@H](C)O)CC2)sc2c(N3CCOCC3)nc(-c3cnc(N)nc3)nc12]

PF-05212384 is a compound of the formula:

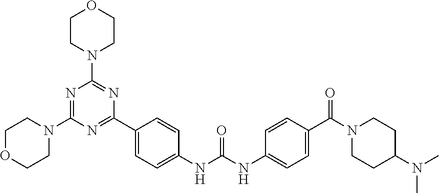 [SMILES: CN(C)C1CCN(C(=O)c2ccc(NC(=O)Nc3ccc(-c4nc(N5CCOCC5)nc(N5CCOCC5)n4)cc3)cc2)CC1]

SAR245409 is a compound of the formula:

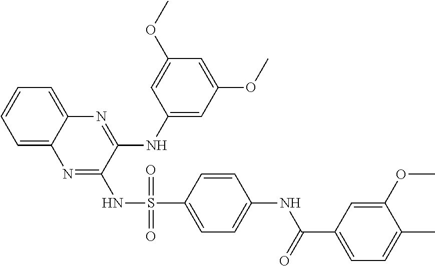 [SMILES: COc1cc(Nc2nc3ccccc3nc2NS(=O)(=O)c2ccc(NC(=O)c3ccc(C)c(OC)c3)cc2)cc(OC)c1]

Compounds that inhibit a polypeptide of the PI3K/AKT/mTOR pathway include, e.g., compounds that target pan-class I PI3K (e.g., BAY80-6946, Buparlisib, Pictilisib, PX-866, SAR245408).

BAY80-6946 is a compound of the formula:

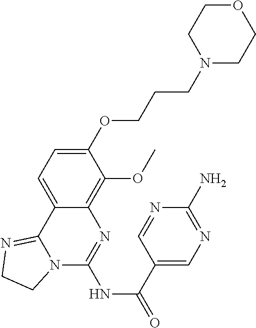 [SMILES: COc1c(OCCCN2CCOCC2)ccc2c1N=C(NC(=O)c1cnc(N)nc1)N1CCN=C21]

Buparlisib is a compound of the formula:

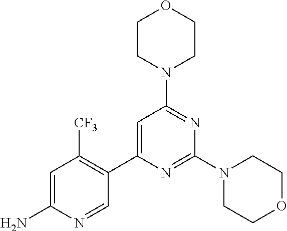 [SMILES: Nc1cc(C(F)(F)F)c(-c2cc(N3CCOCC3)nc(N3CCOCC3)n2)cn1]

Pictilisib is a compound of the formula:

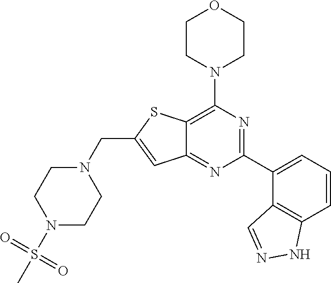 [SMILES: CS(=O)(=O)N1CCN(Cc2cc3nc(-c4cccc5[nH]ncc45)nc(N4CCOCC4)c3s2)CC1]

PX-866 is a compound of the formula:

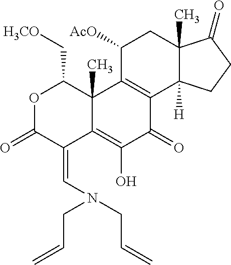 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

SAR245408 is a compound of the formula:

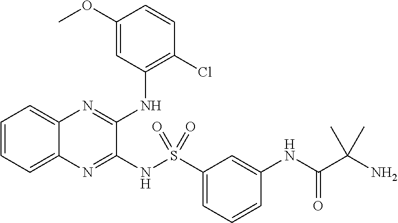 [SMILES: COc1ccc(Cl)c(Nc2nc3ccccc3nc2NS(=O)(=O)c2cccc(NC(=O)C(C)(C)N)c2)c1]

Compounds that inhibit a polypeptide of the PI3K/AKT/mTOR pathway include, e.g., compounds that target PI3K p110α, p110β, p110δ and/or p110γ (e.g., BYL719, GDC-0032, MLN01117, GSK2636771, SAR260301, Idelalisib, AMG319).

BYL719 is a compound of the formula:

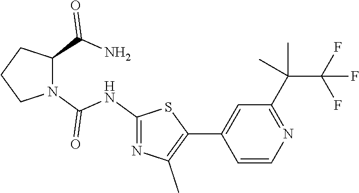 [SMILES: Cc1nc(NC(=O)N2CCC[C@H]2C(N)=O)sc1-c1ccnc(C(C)(C)C(F)(F)F)c1]

GDC-0032 is a compound of the formula:

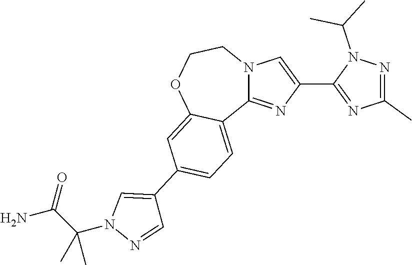 [SMILES: Cc1nc(-c2cn3c(n2)-c2ccc(-c4cnn(C(C)(C)C(N)=O)c4)cc2OCC3)n(C(C)C)n1]

GSK2636771 is a compound of the formula:

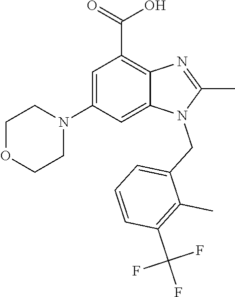 [SMILES: Cc1c(Cn2c(C)nc3c(C(=O)O)cc(N4CCOCC4)cc32)cccc1C(F)(F)F]

SAR260301 is a compound of the formula:

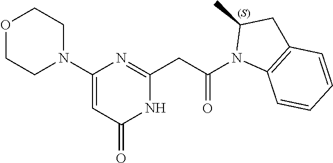

Idelalisib is a compound of the formula:

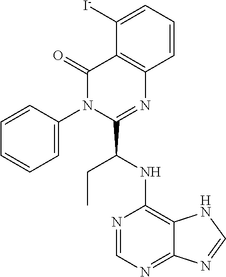 [SMILES: CC[C@H](Nc1ncnc2nc[nH]c12)c1nc2cccc([IH-])c2c(=O)n1-c1ccccc1]

AMG319 is a compound of the formula:

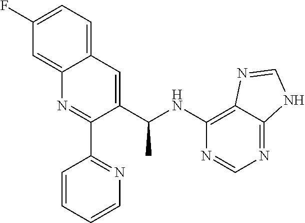 [SMILES: C[C@H](Nc1ncnc2[nH]cnc12)c1cc2ccc(F)cc2nc1-c1ccccn1]

Compounds that inhibit a polypeptide of the PI3K/AKT/mTOR pathway include, e.g., compounds that target AKT (e.g., Perifosine, MK2206, GDC-0068, GSK2110183, GSK2141795, ARQ092, AZD5363).

Perifosine is a compound of the formula:

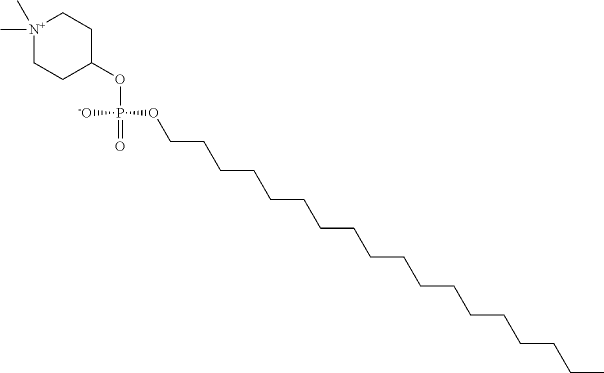

MK2206 is a compound of the formula:

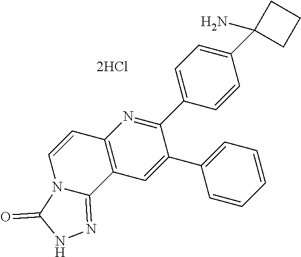 [SMILES: NC1(c2ccc(-c3nc4ccn5c(=O)[nH]nc5c4cc3-c3ccccc3)cc2)CCC1 ; "2HCl"]

GDC-0068 is a compound of the formula:

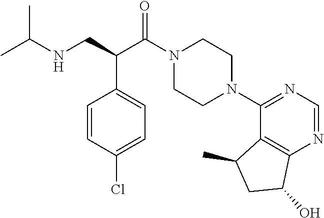 [SMILES: CC(C)NC[C@@H](C(=O)N1CCN(c2ncnc3c2[C@H](C)C[C@H]3O)CC1)c1ccc(Cl)cc1]

GSK2110183 is a compound of the formula:

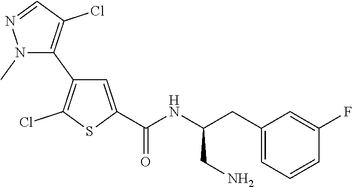 [SMILES: Cn1ncc(Cl)c1-c1cc(C(=O)N[C@H](CN)Cc2cccc(F)c2)sc1Cl]

GSK2141795 is a compound of the formula:

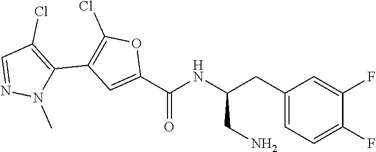 [SMILES: Cn1ncc(Cl)c1-c1cc(C(=O)N[C@H](CN)Cc2ccc(F)c(F)c2)oc1Cl]

AZD5363 is a compound of the formula:

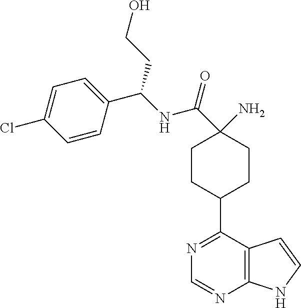 [SMILES: NC1(C(=O)N[C@@H](CCO)c2ccc(Cl)cc2)CCC(c2ncnc3[nH]ccc23)CC1]

Compounds that inhibit a polypeptide of the PI3K/AKT/mTOR pathway include, e.g., compounds that target mTORC1/2 (e.g., AZD2014, MLN0128, CC-223).

AZD2014 is a compound of the formula:

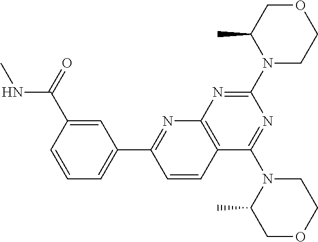 [SMILES: CNC(=O)c1cccc(-c2ccc3c(N4CCOC[C@@H]4C)nc(N4CCOC[C@@H]4C)nc3n2)c1]

MLN0128 is a compound of the formula:

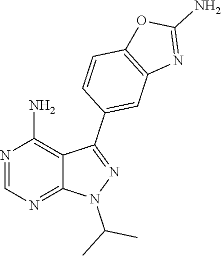 [SMILES: CC(C)n1nc(-c2ccc3oc(N)nc3c2)c2c(N)ncnc21]

As used herein, the combination of a first and second compound of a composition of the present disclosure can provide a synergistic enhancement in inhibiting proliferation of a cancer cell (e.g., melanoma). Accordingly, the combination of the first and second compound inhibits proliferation of a cancer cell more effectively compared to the level of inhibition provided by each single compound alone, or their additive effect when combined. For example, use of I3C in combination with Vemurafenib can provide for a synergistic effect in the inhibition of proliferation of a cancer cell. As another example, use of 1-benzyl I3C in combination with Vemurafenib can provide for a synergistic effect in the inhibition of proliferation of a cancer cell. In some cases, the synergistic effect is provided by two compounds targeting the same protein, e.g., an oncogenic BRAF polypeptide (e.g., BRAF-V600E). In such cases, the two compounds target distinct sites on the oncogenic BRAF polypeptide. In other cases, the synergistic effect can be provided by two compounds targeting different pathways that impinge upon a common downstream component.

Compositions of the present disclosure provide inhibition of proliferation of a cancer cell. In some cases, the cancer cell has acquired drug resistance and cannot be disrupted using conventional therapeutics. For example, melanoma cells acquire resistance against conventional therapeutics, e.g. oncogenic BRAF inhibitors (e.g., Vemurafenib), contributing to the poor long-term prognosis of metastatic melanoma patients. Accordingly, a composition of the present disclosure (e.g., indole-3-carbinol, 1-benzyl indole-3-carbinol, or a pharmaceutically acceptable salt or ester thereof in combination with a second compound (e.g., Vemurafenib)) provides inhibition of proliferation of a melanoma cell that has acquired resistance to treatment by an oncogenic BRAF-inhibitor.

### Dosages

A unit dosage form can be a tablet, a capsule, a unit of liquid volume (e.g., a milliliter, etc.), or any other suitable unit dosage form.

The amount of I3C or 1-benzyl I3C per unit dosage form can range from 0.1 mg to 25 mg, e.g., from 0.1 mg to 0.5 mg, from 0.5 mg to 1 mg, from 1 mg to 1 mg, from 1 mg to 2.5 mg, from 2.5 mg to 5 mg, from 5 mg to 7.5 mg, from 7.5 mg to 10 mg, from 10 mg to 15 mg, from 15 mg to 20 mg, or from 20 mg to 25 mg. The amount of I3C or 1-benzyl I3C per unit dosage form can range from 25 mg to 100 mg, e.g., from 25 mg to 35 mg, from 35 mg to 45 mg, from 45 mg to 55 mg, from 55 mg to 65 mg, from 65 mg to 75 mg, from 75 mg to 85 mg, from 85 mg to 95 mg, from 90 mg to 100 mg. In some cases, the amount of I3C or 1-benzyl I3C per unit dosage form can range from 100 mg to 400 mg, e.g., from 100 mg to 200 mg, from 150 mg to 250 mg, from 200 mg to 300 mg, from 250 mg to 350 mg, from 300 mg to 400 mg, from 350 mg to 450 mg. In some cases the amount of I3C or 1-benzyl I3C per unit dosage form can range from 50 mg to 400 mg.

The amount of Vemurafenib per unit dosage form can range from 100 mg to 1500 mg, e.g., from 100 mg to 200 mg, from 200 mg to 300 mg, from 300 mg to 400 mg, from 400 mg to 500 mg, from 500 mg to 600 mg, from 600 mg to 700 mg, from 700 mg to 800 mg, from 800 mg to 900 mg, from 900 mg to 1000 mg, from 1000 mg to 1100 mg, from 1100 mg to 1200 mg, from 1200 mg to 1300 mg, from 1300 mg to 1400 mg, or from 1400 mg to 1500 mg.

### Formulations

In the subject methods, the active agent(s) may be administered to the host using any convenient means capable of resulting in the desired therapeutic effect or clinical outcome. Thus, an active agent can be incorporated into a variety of formulations for therapeutic administration. More particularly, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. For simplicity, the term “active agent” includes I3C; 1-benzyl-I3C; a compound that binds to and inhibits an oncogenic RAF polypeptide, where the compound binds at a site that is distinct from the site at which the first compound binds; a compound that binds to and inhibits a polypeptide of the RAF signaling pathway; a compound that binds to and inhibits a polypeptide of the Wnt-β-catenin signaling pathway; a compound that binds to and inhibits a polypeptide of the PI3K/AKT/mTOR signaling pathway; and combinations of: i) I3C or 1-benzyl-I3C; and ii) one or more of: a compound that binds to and inhibits an oncogenic RAF polypeptide, where the compound binds at a site that is distinct from the site at which the first compound binds; a compound that binds to and inhibits a polypeptide of the RAF signaling pathway; a compound that binds to and inhibits a polypeptide of the Wnt-β-catenin signaling pathway; a compound that binds to and inhibits a polypeptide of the PI3K/AKT/mTOR signaling pathway.

In pharmaceutical dosage forms, an active agent may be administered in the form of its pharmaceutically acceptable salt, or an active agent may also be used alone or in appropriate association, as well as in combination, with other pharmaceutically active compounds. The following methods and excipients are merely exemplary and are in no way limiting.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. The compounds of the present invention can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise an active agent in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent calculated in an amount sufficient to produce the desired effect in association with a pharmaceutically acceptable diluent, carrier or vehicle. The specifications for a suitable dosage form depend, e.g., on the particular active agent employed and the effect to be achieved, and the pharmacodynamics associated with each compound in the host.

Other modes of administration will also find use with the subject invention. For instance, an active agent can be formulated in suppositories and, in some cases, aerosol and intranasal compositions. For suppositories, the vehicle composition can include traditional binders and carriers such as, polyalkylene glycols, or triglycerides. Such suppositories may be formed from mixtures containing the active ingredient in the range of about 0.5% to about 10% (w/w), e.g., about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of an active agent by the nasal mucosa.

An active agent can be administered in a composition suitable for injection. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Oral Formulations

In some embodiments, an active agent is formulated for oral delivery to an individual in need of such an agent.

For oral delivery, a formulation comprising an active agent will in some embodiments include an enteric-soluble coating material. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™, and shellac.

As one non-limiting example of a suitable oral formulation, an active agent is formulated with one or more pharmaceutical excipients and coated with an enteric coating, as described in U.S. Pat. No. 6,346,269. For example, a solution comprising an active agent and a stabilizer is coated onto a core comprising pharmaceutically acceptable excipients, to form an active agent-coated core; a sub-coating layer is applied to the active agent-coated core, which is then coated with an enteric coating layer. The core generally includes pharmaceutically inactive components such as lactose, a starch, mannitol, sodium carboxymethyl cellulose, sodium starch glycolate, sodium chloride, potassium chloride, pigments, salts of alginic acid, talc, titanium dioxide, stearic acid, stearate, micro-crystalline cellulose, glycerin, polyethylene glycol, triethyl citrate, tributyl citrate, propanyl triacetate, dibasic calcium phosphate, tribasic sodium phosphate, calcium sulfate, cyclodextrin, and castor oil. Suitable solvents for an active agent include aqueous solvents. Suitable stabilizers include alkali-metals and alkaline earth metals, bases of phosphates and organic acid salts and organic amines. The sub-coating layer comprises one or more of an adhesive, a plasticizer, and an anti-tackiness agent. Suitable anti-tackiness agents include talc, stearic acid, stearate, sodium stearyl fumarate, glyceryl behenate, kaolin and aerosil. Suitable adhesives include polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), xanthan gum, alginic acid, salts of alginic acid, Eudragit™, copolymer of methyl acrylic acid/methyl methacrylate with polyvinyl acetate phthalate (PVAP). Suitable plasticizers include glycerin, polyethylene glycol, triethyl citrate, tributyl citrate, propanyl triacetate and castor oil. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™ and shellac.

Suitable oral formulations also include an active agent formulated with any of the following: microgranules (see, e.g., U.S. Pat. No. 6,458,398); biodegradable macromers (see, e.g., U.S. Pat. No. 6,703,037); biodegradable hydrogels (see, e.g., Graham and McNeill (1989) Biomaterials 5:27-36); biodegradable particulate vectors (see, e.g., U.S. Pat. No. 5,736,371); bioabsorbable lactone polymers (see, e.g., U.S. Pat. No. 5,631,015); slow release protein polymers (see, e.g., U.S. Pat. No. 6,699,504; Pelias Technologies, Inc.); a poly(lactide-co-glycolide/polyethylene glycol block copolymer (see, e.g., U.S. Pat. No. 6,630,155; Atrix Laboratories, Inc.); a composition comprising a biocompatible polymer and particles of metal cation-stabilized agent dispersed within the polymer (see, e.g., U.S. Pat. No. 6,379,701; Alkermes Controlled Therapeutics, Inc.); and microspheres (see, e.g., U.S. Pat. No. 6,303,148; Octoplus, B.V.).

Suitable oral formulations also include an active agent formulated with any of the following: a carrier such as Emisphere™. (Emisphere Technologies, Inc.); TIMERx, a hydrophilic matrix combining xanthan and locust bean gums which, in the presence of dextrose, form a strong binder gel in water (Penwest); Geminex™ (Penwest); Procise™ (GlaxoSmithKline); SAVIT™. (Mistral Pharma Inc.); RingCap™ (Alza Corp.); Smartrix®. (Smartrix Technologies, Inc.); SQZgel™ (MacroMed, Inc.); Geomatrix™ (Skye Pharma, Inc.); Oros®. Tri-layer (Alza Corporation); and the like.

Also suitable for use are formulations such as those described in U.S. Pat. No. 6,296,842 (Alkermes Controlled Therapeutics, Inc.); U.S. Pat. No. 6,187,330 (Scios, Inc.); and the like.

Also suitable for use herein are formulations comprising an intestinal absorption enhancing agent. Suitable intestinal absorption enhancers include, but are not limited to, calcium chelators (e.g., citrate, ethylenediamine tetracetic acid); surfactants (e.g., sodium dodecyl sulfate, bile salts, palmitoylcarnitine, and sodium salts of fatty acids); toxins (e.g., zonula occludens toxin); and the like.

### Controlled Release Formulations

In some embodiments, an active agent is formulated in a controlled release formulation.

Controlled release within the scope of this invention can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

The various controlled release technologies cover a very broad spectrum of drug dosage forms. Controlled release technologies include, but are not limited to physical systems and chemical systems.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.).

There are a number of controlled release drug formulations that are developed for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs. Additional information regarding controlled release drug delivery systems may be found in Yie W. Chien, Novel Drug Delivery Systems, 1992 (Marcel Dekker, Inc.). Some of these formulations will now be discussed in more detail.

Enteric coatings are applied to tablets to prevent the release of drugs in the stomach either to reduce the risk of unpleasant side effects or to maintain the stability of the drug which might otherwise be subject to degradation of expose to the gastric environment. Most polymers that are used for this purpose are polyacids that function by virtue or the fact that their solubility in aqueous medium is pH-dependent, and they require conditions with a pH higher than normally encountered in the stomach.

One exemplary type of oral controlled release structure is enteric coating of a solid or liquid dosage form. The enteric coatings are designed to disintegrate in intestinal fluid for ready absorption. Delay of absorption of the active agent that is incorporated into a formulation with an enteric coating is dependent on the rate of transfer through the gastrointestinal tract, and so the rate of gastric emptying is an important factor. In one exemplary embodiment, an active agent can be contained in an enterically coated multiple-unit dosage form. In an exemplary embodiment, a dosage form comprising an active agent is prepared by spray-coating granules of the active agent-enteric coating agent solid dispersion on an inert core material. These granules can result in prolonged absorption of the active agent with good bioavailability.

Typical enteric coating agents include, but are not limited to, hydroxypropylmethylcellulose phthalate, methacryclic acid-methacrylic acid ester copolymer, polyvinyl acetate-phthalate and cellulose acetate phthalate Akihiko Hasegawa, Application of solid dispersions of Nifedipine with enteric coating agent to prepare a sustained-release dosage form, Chem. Pharm. Bull. 33: 1615-1619 (1985). Various enteric coating materials may be selected on the basis of testing to achieve an enteric coated dosage form designed ab initio to have an optimal combination of dissolution time, coating thicknesses and diametral crushing strength. S. C. Porter et al., The Properties of Enteric Tablet Coatings Made From Polyvinyl Acetate-phthalate and Cellulose acetate Phthalate, J. Pharm. Pharmacol. 22:42p (1970).

Another type of useful oral controlled release structure is a solid dispersion. A solid dispersion may be defined as a dispersion of one or more active ingredients in an inert carrier or matrix in the solid state prepared by the melting (fusion), solvent, or melting-solvent method Akihiko Hasegawa, Super Saturation Mechanism of Drugs from Solid Dispersions with Enteric Coating Agents, Chem. Pharm. Bull. 36: 4941-4950 (1998). The solid dispersions may be also called solid-state dispersions. The term “coprecipitates” may also be used to refer to those preparations obtained by the solvent methods.

The selection of the carrier may have an influence on the dissolution characteristics of the dispersed active agent because the dissolution rate of a component from a surface may be affected by other components in a multiple component mixture. For example, a water-soluble carrier may result in a fast release of the drug from the matrix, or a poorly soluble or insoluble carrier may lead to a slower release of the drug from the matrix. The solubility of an active agent may also be increased owing to some interaction with the carriers.

Examples of carriers useful in solid dispersions include, but are not limited to, water-soluble polymers such as polyethylene glycol, polyvinylpyrrolidone, and hydroxypropylmethyl-cellulose. Alternative carriers include phosphatidylcholine. Phosphatidylcholine is an amphoteric but water-insoluble lipid, which may improve the solubility of otherwise insoluble active agents in an amorphous state in phosphatidylcholine solid dispersions.

Other carriers include polyoxyethylene hydrogenated castor oil. Poorly water-soluble active agents may be included in a solid dispersion system with an enteric polymer such as hydroxypropylmethylcellulose phthalate and carboxymethylethylcellulose, and a non-enteric polymer, hydroxypropylmethylcellulose. Another solid dispersion dosage form includes incorporation of an active agent with ethyl cellulose and stearic acid in different ratios.

There are various methods commonly known for preparing solid dispersions. These include, but are not limited to, the melting method, the solvent method and the melting-solvent method.

Another controlled release dosage form is a complex between an ion exchange resin and an active agent. Ion exchange resin-drug complexes have been used to formulate sustained-release products of acidic and basic drugs. In one exemplary embodiment, a polymeric film coating is provided to the ion exchange resin-drug complex particles, making drug release from these particles diffusion controlled. See Y. Raghunathan et al., Sustained-release drug delivery system I: Coded ion-exchange resin systems for phenylpropanolamine and other drugs, J. Pharm. Sciences 70: 379-384 (1981).

Injectable microspheres are another controlled release dosage form. Injectable micro spheres may be prepared by non-aqueous phase separation techniques, and spray-drying techniques. Microspheres may be prepared using polylactic acid or copoly(lactic/glycolic acid). Shigeyuki Takada, Utilization of an Amorphous Form of a Water-Soluble GPIIb/IIIa Antagonist for Controlled Release From Biodegradable Micro spheres, Pharm. Res. 14:1146-1150 (1997), and ethyl cellulose, Yoshiyuki Koida, Studies on Dissolution Mechanism of Drugs from Ethyl Cellulose Microcapsules, Chem. Pharm. Bull. 35:1538-1545 (1987).

Other controlled release technologies that may be used include, but are not limited to, SODAS (Spheroidal Oral Drug Absorption System), INDAS (Insoluble Drug Absorption System), IPDAS (Intestinal Protective Drug Absorption System), MODAS (Multiporous Oral Drug Absorption System), EFVAS (Effervescent Drug Absorption System), PRODAS (Programmable Oral Drug Absorption System), and DUREDAS (Dual Release Drug Absorption System) available from Elan Pharmaceutical Technologies. SODAS are multi particulate dosage forms utilizing controlled release beads. INDAS are a family of drug delivery technologies designed to increase the solubility of poorly soluble drugs. IPDAS are multi particulate tablet formation utilizing a combination of high density controlled release beads and an immediate-release granulate. MODAS are controlled release single unit dosage forms. Each tablet consists of an inner core surrounded by a semipermeable multiparous membrane that controls the rate of drug release. EFVAS is an effervescent drug absorption system. PRODAS is a family of multi particulate formulations utilizing combinations of immediate release and controlled release mini-tablets. DUREDAS is a bilayer tablet formulation providing dual release rates within the one dosage form. Although these dosage forms are known to one of skill, certain of these dosage forms will now be discussed in more detail.

INDAS was developed specifically to improve the solubility and absorption characteristics of poorly water soluble drugs. Solubility and, in particular, dissolution within the fluids of the gastrointestinal tract is a key factor in determining the overall oral bioavailability of poorly water soluble drug. By enhancing solubility, one can increase the overall bioavailability of a drug with resulting reductions in dosage. INDAS takes the form of a high energy matrix tablet, production of which is comprised of two distinct steps: the drug in question is converted to an amorphous form through a combination of energy, excipients, and unique processing procedures.

Once converted to the desirable physical form, the resultant high energy complex may be stabilized by an absorption process that utilizes a novel polymer cross-linked technology to prevent recrystallization. The combination of the change in the physical state of an active agent coupled with the solubilizing characteristics of the excipients employed enhances the solubility of the active agent. The resulting absorbed amorphous drug complex granulate may be formulated with a gel-forming erodible tablet system to promote substantially smooth and continuous absorption.

IPDAS is a multi-particulate tablet technology that may enhance the gastrointestinal tolerability of potential irritant and ulcerogenic drugs. Intestinal protection is facilitated by the multi-particulate nature of the IPDAS formulation which promotes dispersion of an irritant lipoate throughout the gastrointestinal tract. Controlled release characteristics of the individual beads may avoid high concentration of drug being both released locally and absorbed systemically. The combination of both approaches serves to minimize the potential harm of an active agent with resultant benefits to patients.

IPDAS is composed of numerous high density controlled release beads. Each bead may be manufactured by a two-step process that involves the initial production of a micromatrix with embedded active agent and the subsequent coating of this micromatrix with polymer solutions that form a rate-limiting semipermeable membrane in vivo. Once an IPDAS tablet is ingested, it may disintegrate and liberate the beads in the stomach. These beads may subsequently pass into the duodenum and along the gastrointestinal tract, e.g., in a controlled and gradual manner, independent of the feeding state. Release of the active agent occurs by diffusion process through the micromatrix and subsequently through the pores in the rate controlling semipermeable membrane. The release rate from the IPDAS tablet may be customized to deliver a drug-specific absorption profile associated with optimized clinical benefit. Should a fast onset of activity be necessary, immediate release granulate may be included in the tablet. The tablet may be broken prior to administration, without substantially compromising drug release, if a reduced dose is required for individual titration.

MODAS is a drug delivery system that may be used to control the absorption of water soluble agents. Physically MODAS is a non-disintegrating table formulation that manipulates drug release by a process of rate limiting diffusion by a semipermeable membrane formed in vivo. The diffusion process essentially dictates the rate of presentation of drug to the gastrointestinal fluids, such that the uptake into the body is controlled. Because of the minimal use of excipients, MODAS can readily accommodate small dosage size forms. Each MODAS tablet begins as a core containing active drug plus excipients. This core is coated with a solution of insoluble polymers and soluble excipients. Once the tablet is ingested, the fluid of the gastrointestinal tract may dissolve the soluble excipients in the outer coating leaving substantially the insoluble polymer. What results is a network of tiny, narrow channels connecting fluid from the gastrointestinal tract to the inner drug core of water soluble drug. This fluid passes through these channels, into the core, dissolving the drug, and the resultant solution of drug may diffuse out in a controlled manner. This may permit both controlled dissolution and absorption. An advantage of this system is that the drug releasing pores of the tablet are distributed over substantially the entire surface of the tablet. This facilitates uniform drug absorption reduces aggressive unidirectional drug delivery. MODAS represents a very flexible dosage form in that both the inner core and the outer semipermeable membrane may be altered to suit the individual delivery requirements of a drug. In particular, the addition of excipients to the inner core may help to produce a microenvironment within the tablet that facilitates more predictable release and absorption rates. The addition of an immediate release outer coating may allow for development of combination products.

Additionally, PRODAS may be used to deliver an active agent. PRODAS is a multi particulate drug delivery technology based on the production of controlled release mini tablets in the size range of 1.5 to 4 mm in diameter. The PRODAS technology is a hybrid of multi particulate and hydrophilic matrix tablet approaches, and may incorporate, in one dosage form, the benefits of both these drug delivery systems.

In its most basic form, PRODAS involves the direct compression of an immediate release granulate to produce individual mini tablets that contain an active agent. These mini tablets are subsequently incorporated into hard gels and capsules that represent the final dosage form. A more beneficial use of this technology is in the production of controlled release formulations. In this case, the incorporation of various polymer combinations within the granulate may delay the release rate of drugs from each of the individual mini tablets. These mini tablets may subsequently be coated with controlled release polymer solutions to provide additional delayed release properties. The additional coating may be necessary in the case of highly water soluble drugs or drugs that are perhaps gastroirritants where release can be delayed until the formulation reaches more distal regions of the gastrointestinal tract. One value of PRODAS technology lies in the inherent flexibility to formulation whereby combinations of mini tablets, each with different release rates, are incorporated into one dosage form. As well as potentially permitting controlled absorption over a specific period, this also may permit targeted delivery of drug to specific sites of absorption throughout the gastrointestinal tract. Combination products also may be possible using mini tablets formulated with different active ingredients.

DUREDAS is a bilayer tableting technology that may be used to an active agent. DUREDAS was developed to provide for two different release rates, or dual release of a drug from one dosage form. The term bilayer refers to two separate direct compression events that take place during the tableting process. In an exemplary embodiment, an immediate release granulate is first compressed, being followed by the addition of a controlled release element which is then compressed onto this initial tablet. This may give rise to the characteristic bilayer seen in the final dosage form.

The controlled release properties may be provided by a combination of hydrophilic polymers. In certain cases, a rapid release of an active agent may be desirable in order to facilitate a fast onset of therapeutic effect. Hence one layer of the tablet may be formulated as an immediate release granulate. By contrast, the second layer of the tablet may release the drug in a controlled manner, e.g., through the use of hydrophilic polymers. This controlled release may result from a combination of diffusion and erosion through the hydrophilic polymer matrix.

A further extension of DUREDAS technology is the production of controlled release combination dosage forms. In this instance, two different active agents may be incorporated into the bilayer tablet and the release of drug from each layer controlled to maximize therapeutic effect of the combination.

An active agent can be incorporated into any one of the aforementioned controlled released dosage forms, or other conventional dosage forms. The amount of active agent contained in each dose can be adjusted, to meet the needs of the individual patient, and the indication. One of skill in the art and reading this disclosure will readily recognize how to adjust the level of an active agent and the release rates in a controlled release formulation, in order to optimize delivery of the active agent and its bioavailability.

### Methods of Treatment

The present disclosure provides a method of treating cancer (e.g., melanoma) in a subject, the method comprising administering to the subject an effective amount of a first compound selected from I3C, 1-benzyl I3C, or a pharmaceutically acceptable salt or ester thereof, and a second compound that binds to and inhibits: i) an oncogenic RAF polypeptide at a site that is distinct from the site at which the first compound binds; ii) a polypeptide of the RAF signaling pathway; iii) a polypeptide of the Wnt-β-catenin signaling pathway; iv) or a polypeptide of the PI3K/AKT/mTOR signaling pathway. In some cases, the first compound and the second compound are administered in the same formulation. In some cases, the first compound and the second compound are administered in separate formulations. In some cases, the first compound and the second compound are administered substantially simultaneously. In some cases, the first compound and the second compound are administered within 1 hour to 24 hours of one another.

The present disclosure provides a method of treating cancer in a subject comprising administering to the subject an effective amount of a composition as described above. The present disclosure provides a method of treating cancer in a subject comprising administering to the subject an effective amount of: a) a first compound selected from indole-3-carbinol, 1-benzyl indole-3-carbinol, or a pharmaceutically acceptable salt or ester thereof; and b) a second compound that binds an oncogenic BRAF polypeptide at a site that is distinct from the site at which the first compound binds. In some cases, the method comprises administering 1-benzyl-I3C (first compound) and Vemurafenib (second compound). In some cases, the first compound and the second compound are administered in the same formulation. In some cases, the first compound and the second compound are administered in separate formulations. In some cases, the first compound and the second compound are administered substantially simultaneously. In some cases, the first compound and the second compound are administered within 1 hour to 24 hours of one another.

For simplicity, the term “agent,” or “active agent,” as used herein, includes I3C; 1-benzyl-I3C; a compound that binds to and inhibits an oncogenic RAF polypeptide, where the compound binds at a site that is distinct from the site at which the first compound binds; a compound that binds to and inhibits a polypeptide of the RAF signaling pathway; a compound that binds to and inhibits a polypeptide of the Wnt-β-catenin signaling pathway; a compound that binds to and inhibits a polypeptide of the PI3K/AKT/mTOR signaling pathway; and combinations of: i) I3C or 1-benzyl-I3C; and ii) one or more of: a compound that binds to and inhibits an oncogenic RAF polypeptide, where the compound binds at a site that is distinct from the site at which the first compound binds; a compound that binds to and inhibits a polypeptide of the RAF signaling pathway; a compound that binds to and inhibits a polypeptide of the Wnt-β-catenin signaling pathway; a compound that binds to and inhibits a polypeptide of the PI3K/AKT/mTOR signaling pathway.

### Dosages

A suitable dosage can be determined by an attending physician or other qualified medical personnel, based on various clinical factors. As is well known in the medical arts, dosages for any one patient depend upon many factors, including the patient's size, body surface area, age, the particular compound to be administered, sex of the patient, time, and route of administration, general health, and other drugs being administered concurrently. An agent can be administered in amounts between 1 ng/kg body weight and 20 mg/kg body weight per dose, e.g., between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. In some cases, a suitable dosage can be administered to a patient once a day, or multiple times a day as determined by an attending physician, e.g., twice, three times, four times, five times, six times, at least twice a day.

In some cases, a first compound and a second compound are administered to a subject in need thereof in the same formulation. In some cases, a first compound and a second compound are administered to a subject in need thereof in separate formulations and substantially simultaneously. In some cases, a first compound and a second compound are administered to a subject in need thereof in separate formulations and within about 1 week, within about 5 days, within about 3 days, within about 1 day, within about 12 hours, within about 8 hours, within about 4 hours, within about 2 hours, or within about 1 hour, of one another.

The amount of Vemurafenib per dose can range from 100 mg to 1500 mg, e.g., from 100 mg to 200 mg, from 200 mg to 300 mg, from 300 mg to 400 mg, from 400 mg to 500 mg, from 500 mg to 600 mg, from 600 mg to 700 mg, from 700 mg to 800 mg, from 800 mg to 900 mg, from 900 mg to 1000 mg, from 1000 mg to 1100 mg, from 1100 mg to 1200 mg, from 1200 mg to 1300 mg, from 1300 mg to 1400 mg, or from 1400 mg to 1500 mg. In some cases, Vemurafenib is administered in an amount of about 960 mg twice daily.

The amount of 1-benzyl I3C per dose can range from 0.1 mg to 25 mg, e.g., from 0.1 mg to 0.5 mg, from 0.5 mg to 1 mg, from 1 mg to 1 mg, from 1 mg to 2.5 mg, from 2.5 mg to 5 mg, from 5 mg to 7.5 mg, from 7.5 mg to 10 mg, from 10 mg to 15 mg, from 15 mg to 20 mg, or from 20 mg to 25 mg.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

### Routes of Administration

An active agent is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, intracranial, subcutaneous, intradermal, topical application, intravenous, rectal, nasal, oral and other enteral and parenteral routes of administration. Routes of administration may be combined, if desired, or adjusted depending upon the agent and/or the desired effect. The composition can be administered in a single dose or in multiple doses. In some embodiments, the composition is administered orally. In other cases, the composition is administered intravenously. In other cases, the composition is administered via an inhalational route. In other cases, the composition is administered intramuscularly.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as a neurological disorder and pain that may be associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

The embodiments include compositions comprising a container suitable for containing a composition of the present disclosure for administration to an individual. For example, a subject composition can be disposed within a container suitable for containing a pharmaceutical composition. The container can be, for example, a bottle (e.g., with a closure device, such as a cap), a blister pack (e.g., which can provide for enclosure of one or more doses per blister), a vial, flexible packaging (e.g., sealed Mylar or plastic bags), an ampule (for single doses in solution), a dropper, a syringe, thin film, a tube and the like. In some cases, a container, such as a sterile container, comprises a subject pharmaceutical composition. In some embodiments the container is a bottle or a syringe. In some embodiments the container is a bottle. In some embodiments the container is a syringe.

Kits with unit doses of a subject composition, e.g. in oral or topical doses, are provided. In such kits, in addition to the containers containing the unit doses will be an informational package insert describing the use and attendant benefits of the composition in treating pathological condition of interest. Preferred compounds and unit doses are those described herein above.

### Subjects Suitable for Treatment

A variety of subjects (wherein the term “subject” is used interchangeably herein with the terms “host” and “patient”) are treatable according to the methods of the present disclosure. Generally such subjects are “mammals” or “mammalian,” where these terms are used broadly to describe organisms which are within the class mammalia, including the orders carnivore (e.g., dogs and cats), rodentia (e.g., mice, guinea pigs, and rats), non-human primates, and primates (e.g., humans, chimpanzees, and monkeys). In some cases, a suitable subject for treatment methods of the present disclosure is a human.

Subjects suitable for treatment with a subject method include individuals who have been diagnosed as having cancer. The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for treatment in the present disclosure include precancerous (e.g., benign), malignant, pre-metastatic, metastatic, and non-metastatic cells. Treatment of cancerous cells is of particular interest. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc. Cancers of interest include, without limitation, hematopoietic cancers including leukemias, lymphomas (Hodgkins and non-Hodgkins), myelomas and myeloproliferative disorders; sarcomas, melanomas, adenomas, carcinomas of solid tissue, squamous cell carcinomas of the mouth, throat, larynx, and lung, liver cancer, genitourinary cancers such as cervical, bladder cancer and renal cell carcinomas, head and neck cancers, gastro intestinal track cancers and nervous system cancers, benign lesions such as papillomas, and the like.

In some cases, subjects suitable for treatment using methods of the present disclosure include individuals diagnosed with melanoma, e.g., superficial spreading melanoma, lentigo maligna melanoma, acral lentiginous melanoma, nodular melanoma. In some cases, subjects suitable for treatment using methods of the present disclosure include individuals diagnosed with drug resistant melanoma (e.g., malignant melanoma). In some cases, cells of individuals diagnosed with drug resistant melanoma harbor oncogenic BRAF mutations (e.g., BRAF-V600E mutation). In such cases, such individuals have been diagnosed with BRAF-inhibitor resistant melanoma, and are suitable for treatment using methods of the present disclosure.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Materials and Methods

### Cell Culture

Melanoma cell lines G-361, SK-MEL-28, SK-MEL-2, SK-MEL-30 and RPMI-7951 and melanocytes were purchased from American Type Culture Collection (ATCC) (Manasas, Va.), and were authenticated according to the ATCC guidelines. DM738 melanoma cells were acquired from the tissue culture facility at University of California, Berkeley. The G361 melanoma cells were cultured in Modified McCoy's 5A cell media supplemented with 10% fetal bovine serum (Gemini Bio Products), 2 mM L-glutamine, and 2.5 ml of 10,000 U/ml penicillin/streptomycin mixture (Gibco, Life Technologies, Carlsbad, Calif.). DM738, SK-MEL-28 and SK-MEL-30 cells were cultured in DMEM with 4.5 g/L glucose and 2 mM L-glutamine supplemented with 10% fetal bovine serum, 2.5 ml of 10,000 U/ml penicillin/streptomycin mixture in addition to 1× of MEM Non-Essential Amino Acid (Gibco, Life Technologies). RPMI-7951 and SK-MEL-2 melanoma cells were cultured in DMEM containing 4.5 g/L Glucose, 114 mg/L Sodium Pyruvate, and 2 mM L-glutamine, supplemented as described above. The cells were incubated in tissue culture dishes (Nalgene Nunc) at 37° C. with controlled humidity and 5% CO₂ air content.

Treatment with 1-benzyl Indole-3-carbinol, Indole-3-carbinol, Vemurafenib, Lithium Chloride (LiCl), 6-bromoindirubin-3′-oxime (BIO).

I3C and BIO was purchased from (Sigma Aldrich), Vemurafenib from (Adooq Bioscience), and LiCl from (Fischer Scientific). To study the effect of 1-benzyl I3C in comparison to its parent compound I3C and the commercial drug Vemurafenib, cells were treated with 1, 5, 10, 20, or 25 μM of 1-benzyl I3C, 50, 100, 200, 300 μM I3C and 1, 5, 10, 15 μM Vemurafenib every 24 hours for 48 hours and CCK-8 proliferation assay was performed on the cells at the end of the treatment. The same treatment regime was repeated on multiple melanoma cell lines with 20 μM 1-benzyl I3C and 200 μM I3C for 48 hours to compare the relative sensitivities of the different cell lines with distinct mutation profiles to 1-benzyl I3C and I3C. Cells were treated with 20 μM 1-benzyl I3C every 24 hours for 72 hours and harvested at 24, 48 and 72 hours for western blot and flow cytometric analysis. Cells were treated with 20 μM LiCl or 10 μM BIO for 48 hours before harvesting them for western blots. For the experiments on the combination of 1-benzyl I3C and Vemurafenib treatment, cells were treated with the compounds for 24 hours. I3C, Vemurafenib, LiCl and BIO were dissolved in 99.9% high performance liquid chromatography (HPLC) grade dimethylsulfoxide (DMSO) (Sigma Aldrich, Milwaukee, Wis.) and the final dilution was performed in the media aliquots used for treatment.

Treatment with Indole-3-carbinol, Vemurafenib and UO126

I3C was purchased from Sigma Aldrich (St. Louis, Mo.), Vemurafenib (Adooq Bioscience) and UO126 (Cell Signaling). To study the effect of I3C, cells were treated with or without 200 μM I3C every 24 hours for 72 hours and harvested at 24, 48 and 72 hours for western blot and flow cytometric analysis. Cells were treated with Vemurafenib and UO126 for 48 hours before harvesting them for western blots. For the experiments on the combination of I3C and Vemurafenib treatment, cells were treated with the compounds for 24 hours. I3C, Vemurafenib and UO126 were dissolved in 99.9% HPLC grade DMSO (Sigma Aldrich, Milwaukee, Wis.) and the final dilution was performed in the media aliquots used for treatment.

### MTT Proliferation Assay

Melanoma cell lines were seeded on a 48 well plate and upon 80-90% confluency were either treated in triplicates with different concentrations of 1-benzyl I3C, I3C or Vemurafenib as well as combinations of 1-benzyl I3C and Vemurafenib or DMSO for time durations specified for each experiment. Subsequently inhibition of proliferation was assessed using Cell counting Kit-8 (Dojindo) as per the protocol in the user's manual. Briefly, 50 μl of the CCK-8 solution was added to each well along with 450 μl media and incubated for 2-3 hours. The absorbance was read at 450 nm and % inhibition was calculated for each condition standardizing DMSO to zero using the formula: [(100−Value of treatment/Value of DMSO treated control)×100]

### In Vitro BRAF Kinase Assay

BRAF immunoprecipitations were carried out either using G-361 cells treated with DMSO, I3C or Vemurafenib for 72 hours or from untreated G-361 and SK-MEL-2 cells as described previously (Aronchik et al., Cancer Res. 2010. 70:4961-4971). BRAF was pulled down from the cell lysates using anti-BRAF antibodies bound to protein G-coupled Sepharose beads, while IgG was used as a negative control. Post-immunoprecipitation supernatants were probed with anti BRAF antibody by western blots to determine pull-down efficiency. Only in the “In-Vitro treated” samples DMSO, I3C and Vemurafenib was added to the immunoprecipitated wild type and mutant BRAF prior to the kinase assay, and reactions were incubated at 30° C. for 2 hours. Kinase assay was subsequently performed with the immunoprecipitated wild type or oncogenic BRAF from the pre-treated as well as the “in-vitro treated” samples by incubation with recombinant inactive MEK(K97R) (Millipore) supplemented with ATP/Mg²⁺ in kinase reaction buffer at 30° C. for 30 mins. The reactions were terminated by adding 4× sodium dodecyl sulfate (SDS) sample buffer and boiling the mixture at 100° C. for 5 min. The samples were then electrophoresed, immunoblotted and probed for relative protein levels of phosphorylated MEK and total MEK to assess the kinase activity of the immunoprecipitated BRAF.

### In Silico Modeling

The structures of the WT BRAF and V600E BRAF proteins were obtained from the Protein Data Bank (PDB) with accession numbers 3Q4C and 3OG7 respectively. The PRODRG server was used to produce the topology files for modeling the I3C structure. The proteins (3Q4C & 3OG7) and ligand (I3C) structures were subsequently loaded into the Hex Protein Docking program. Prior to docking the structures, all water molecules and hetero molecules were manually removed by editing each PDB file. Shape and electrostatics were used as restrictive parameters to model binding between the receptor and ligand. Modeling results were visualized using PyMol. The program LigPlot was then used to generate schematic diagrams that illustrate the pattern of interactions between the 3-D coordinates of the protein and bound ligand.

### Flow Cytometry Analysis of DNA Content

Melanoma cells were treated with the indicated concentrations of 1-benzyl I3C in triplicates every 24 hours and harvested at the end of 24, 48 or 72 hours for cell cycle analysis. The DNA content of propidium iodide stained nuclei from harvested cells were determined by flow cytometry. Briefly, cells were hypotonically lysed in 300 mL of DNA staining solution (0.5 mg/mL propidium iodide, 0.1% sodium citrate, and 0.05% Triton-X 100). Emitted fluorescence from the nuclear of wavelengths more than 585 nm was measured with a Coulter Elite instrument with laser output adjusted to deliver 15 mW at 488 nm. Ten thousand nuclei were analyzed from each sample at a rate of 300-500 nuclei/second. The percentage of cells within the G₁, S, and G₂/M phases of the cell cycle were determined by analysis with the Multicycle computer program provided by Phoenix Flow Systems in the Cancer Research Laboratory Microchemical Facility of the University of California, Berkeley.

### Western Blot Analysis

Western Blot analyses of samples electrophoretically fractionated on 12% acrylamide gels were carried out as previously described (Wagle et al., J. Clin. Oncol. 2011. 29(22):3085-3096; Nguyen et al., Chem. Biol. Interact. 2010. 186(3):255-266). Enhanced chemiluminescence (ECL) Lightening reagents were used to visualize the primary antibody bound protein bands in nitrocellulose membranes and the results captured on ECL Autoradiography Film (GE Healthcare, Piscataway, N.J.). The western blots employed the following primary antibodies: mouse anti-MITF-M (Thermo-scientific), mouse anti-HSP 90 (BD Biosciences), mouse anti-CDK2, rabbit anti-CDK4, rabbit anti-CDK-6, mouse anti-rabbit BRAF, goat anti-LEF-1, rabbit anti-AXIN, mouse anti-BCL2, (Santa Cruz), rabbit anti-P21, mouse anti-Cyclin D1, rabbit anti-MEK-p, rabbit anti-MEK1/2, rabbit anti-GSK3β rabbit anti-LRP6, rabbit anti-LRP6-phospho (Cell signaling), mouse anti-β-catenin (Invitrogen).

### RT-PCR Analysis

Total RNA was extracted from harvested cells using the RNeasy extraction kit for mammalian cells (obtained from Qiagen, Hercules, Calif.) and spectrophotometrically quantified by absorbance at 260 nm. Reverse transcription (RT) reactions were carried out using RT-MMLV reverse transcriptase (Invitrogen, Eugene, Oreg.) and the cDNA was used for polymerase chain reaction (PCR) reactions using 10 pM of the following primers: MITF-M forward 5′ CCG TCT CTC ACT GGA TTG GT 3′ (SEQ ID NO:1), MITF-M Reverse 5′ TAC TTG GTG GGG TTT TCG AG 3′ (SEQ ID NO:2) GAPDH forward, TGAACGGGAAGCTCACTGG (SEQ ID NO:3) and reverse, TCCACCACCCTGTTGCTGTA (SEQ ID NO:4). PCR conditions were as follows: 30 s at 94° C., 30 s at 55° C. and 30 s at 72° C. for 28 cycles. PCR products were electrophoretically fractionated in 1.5% agarose gels containing 0.01% Gel Red (Biotium) for DNA staining along with 1 kb plus DNA ladder (Thermo Scientific) and further visualized by a UV transilluminator.

### Tumor Xenografts and Immunofluorescence

Approximately 1 million G361 melanoma cells in a total volume of 0.1 ml of Matrigel were injected subcutaneously into each lateral flank of NIH III athymic nude mice. The resulting tumors were allowed to grow to a mean starting volume of 146±10 mm³. The animals were then randomized into two groups; a vehicle control group that was treated with DMSO and an I3C treated group that were injected daily with 200 mg/kg body weight of I3C. The resulting tumor volumes were measured every other day for four weeks using calipers. The tumor volumes were calculated using the standard formula: (Width×Length)/2, and changes in tumor volumes were calculated using the formula: 100+{(Tf−Ti)/Ti×100}, where Tf is the final mean tumor volume and Ti is the initial mean tumor volume. At terminal sacrifice, tumor xenografts were harvested, and a portion of each tumor was fixed in 4% para-formaldehyde for 1 hour at room temperature, followed by a phosphate-buffered saline (PBS) wash and subsequently immersed in 3% sucrose overnight at 4° C. The resulting tissues were embedded in OCT and 10 μm thin sections were taken for immunofluorescence studies.

### Luciferase Reporter Assay

G361 Melanoma cells were cultured to approximately 80% confluency in 6 well adherent culture plates in triplicates for each experimental condition for 24 hours. The cells were then transiently transfected with either a wild-type pGL2-pMITF-333/+120-luciferase reporter plasmid or a similar plasmid with the LEF-1/TCF binding site mutated at −199. pGL2-pMITF-333/+120-ΔLEF-1-luciferase, (ALEF-1) was generated using the following primers: LEF-1 forward, 5′-GACAGTGAGTTTGACTTTGGCAGCTCGTCACTTAA-3′ (SEQ ID NO:5) and LEF-1 reverse, 5′-TTAAGTGACGAGCTGCCAAAGTCAAACTCACTGTC-3′ (SEQ ID NO:6).

PCR conditions used were as follows: 30 s hotstart at 95° C., 30 s at 95° C., 1 min at 55° C., 8 min at 68° C. for 16 cycles. Mutagenesis was performed using QuickChange II kit (Aligent) per the manufacturer's instructions. PCR products were extracted and purified using QIAquick Gel Extraction Kit (Qiagen). Sequence was confirmed by automated DNA sequencing (University of California Berkeley Sequencing Facility). SuperFect Transfection Reagent (Qiagen) was used for the transfections as per instructions in the user manual. The next day the cells were either treated with 200 μM I3C or an equal volume of the DMSO vehicle control for both wild-type plasmid transfected as well as mutant plasmid transfected cells. 24 hours later luciferase assays were performed by harvesting cells in 1× Promega Lysis Buffer, allowing it to incubate for 15 minutes at room temperature, followed by 15 minutes incubation on ice. The cells were then pelleted by a spin at 15,000 rpm for 1 minute at 4° C. 20 μl of supernatant from each sample was combined with 100 μl luciferase substrate (Promega) and emitted fluorescence was measured using luminometer, Lumat LB 9507 (EG&G Berthold) and the relative light units (RLU) were recorded. The RLU/μg for each sample was calculated by dividing the data obtained with their specific protein concentration determined by a Bradford assay using 1× BioRad Protein Assay reagent and measuring O.D at 595 nm.

### Chromatin Immunoprecipitation

Chromatin immunoprecipitation was performed using G361 melanoma cells treated with or without 200 μM I3C for 72 hours. Proteins were crosslinked to DNA by the addition of formaldehyde at 2.5% of the final concentration for 5 minutes at room temperature to cultured cells. Fixation was quenched with glycine for a final concentration of 125 mmol/1 for 5 minutes. Harvested cells were sonicated and the chromatin was immunoprecipitated overnight and 15 μl of mouse anti-Brn2 antibodies (Santa Cruz Biotechnology) was added to the cell lysates to immunoprecipitate the chromatin-bound Brn2 protein. A fragment of the MITF-M promoter (−170+120) containing the BRN2 binding site (−53 to −27) was amplified using the primers—MITF-M Forward: 5′-CGT CAC TTA AAA AGG TAC CTT TAT ATT TAT G-3′ (SEQ ID NO:7) MITF-M Reverse: 5′-TGT TTTAGC TAG CAC CAA TCC AGT GAG AGA CGG-3′ (SEQ ID NO:8) by cycling 36 times (95° C., 30 s/52° C. 30 s/72° C., 30 s) with a 72° C., 10 min extension. The PCR products were electrophoretically fractionated on a 1.5% agarose gel and visualized using a transilluminator.

### Wnt Conditioned Media

Murine fibroblast L cells stably transfected with a construct expressing high levels of human Wnt3A were used. The cells were grown in DMEM containing 4.5 g/L Glucose, 114 mg/L Sodium Pyruvate, and 2 mM L-glutamine, supplemented with with 10% fetal bovine serum (Gemini Bio Products), 2 mM L-glutamine, and 2.5 ml of 10,000 U/ml penicillin/streptomycin mixture (Gibco, Life Technologies). Conditioned media was harvested in two batches, once during routine media change after 2 days of seeding the cells and once more when the plates were confluent. The cells were trypsinized as described earlier using 0.025% trypsin (Lonza) and pelleting the cells by a spin at 4000 rpm for 5 minutes and aspirating and filtering the supernatant through a 0.2 micron filter to obtain the conditioned media. The two batches of conditioned media were mixed 1:1 and stored in aliquots at −80° C. for future use.

### TOP Flash Reporter Assay

The TOPflash/FOPflash (Millipore) reporter assay was performed on G361 cells to assess the effect of 1-benzyl I3C on β-catenin driven Wnt transcriptional activity. TOPflash is a TCF/LEF responsive luciferase construct encoding the firefly luciferase reporter under the control of a minimal (m) CMV promoter and tandem repeats of a TCF/LEF transcriptional response element (TRE). FOPflash is a similar construct with the LEF-1 binding site mutated which serves as the negative control. G361 cells were cultured to approximately 80% confluency in a 6 well clear bottom plate (Nunc). The TOPflash/FOPflash construct was transfected using Superfect Transfection Reagent (Qiagen.) according to the manufactures instructions. Cells in each well were transfected with 2 μg DNA and 4 μl Superfect for 2-3 hours and then replaced with 2 ml of 1:1 ratio of fresh and conditioned media from cultured L-cells secreting Wnt or no Wnt. 24 hours later the cells were treated with/without 10 μM 1-benzyl I3C for another 24 hours in conditioned media. Cells were washed with DPBS, resuspended in Lysis Buffer (Promega), incubated on ice for 15 minutes, spun at 14000 rpm for 1 minute at 4° C. and the supernatant is used for detection of luciferase activity. 20 μl of supernatant from each sample was combined with 100 μl luciferase substrate (Promega) and emitted fluorescence was measured using luminometer, Lumat LB 9507 (EG&G Berthold) and the relative light units (RLU) were recorded. The RLU/μg for each sample was calculated by dividing the data obtained with their specific protein concentration determined by a Bradford assay using 1× BioRad Protein Assay reagent and measuring O.D at 595 nm. The experiment was performed in triplicates at least 3 times.

### Example 1: I3C Inhibits Cellular and In Vivo Proliferation and Down-Regulates MITF in BRAF-V600E Expressing Melanoma Cells

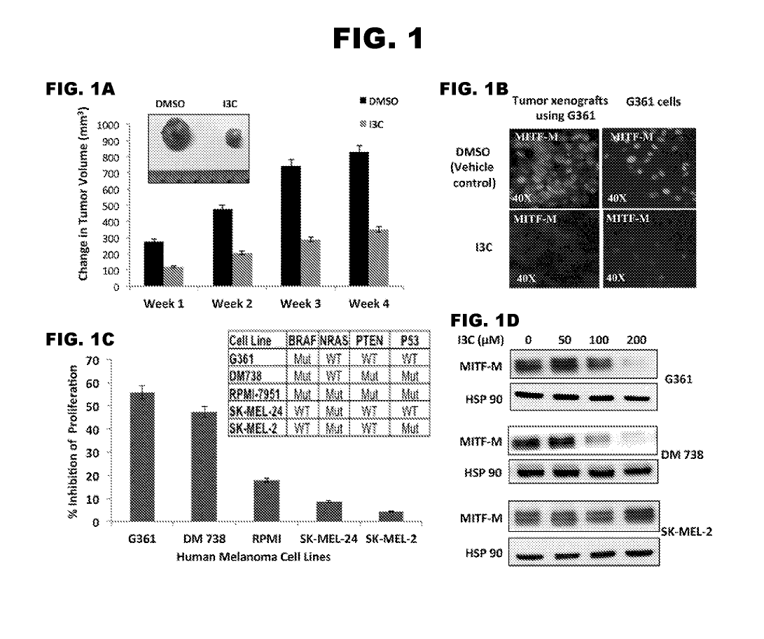

Because the melanocyte master regulator Micropthalmia Associated Transcription Factor (MITF-M) plays a central role in the regulation of melanoma cell proliferation and differentiation (Yajima et al., J. Skin Cancer 2011. 2011:730170), it was examined whether indole-3-carbinol (I3C) disrupts the in vivo production of this transcription factor in human melanoma cell-derived tumor xenografts was examined. G-361 melanoma cells expressing oncogenic BRAFV600E and sensitive to the anti-proliferative effects of I3C (Aronchik et al., Mol. Cancer Res. 2014. 12:1621-1634) were subcutaneously injected into NIH III athymic mice to generate xenografted tumors. The mice were injected with either I3C (200 mg/kg body weight) or DMSO vehicle control throughout a four-week course. I3C strongly inhibited tumor growth that was noticeable within the first week of injections and sustained over the entire time course (FIG. 1A). Immunofluorescence analysis of tumor xenograft sections demonstrated that concomitant with the in vivo inhibition of tumor growth, I3C injected animals showed a strong down-regulation of MITF-M protein relative to vehicle treated control animals (FIG. 1B, left panels). Similar down regulation of MITF-M protein was observed by immunofluorescence analysis of cultured G-361 melanoma cells treated for 48 hours with or without 200 μM I3C (FIG. 1B, right panels), which is the optimal I3C concentration for the anti-proliferative effect in cultured human cancer cells (Wong et al., J. Cell Biochem. Suppl. 1997. 28-29:111-116). This result indicated that the I3C down regulation of MITF-M levels observed in vivo is a direct effect in the melanoma cells, rather than an indirect consequence of other tissues conceivably producing a factor in response to I3C that acts on the melanoma-derived tumor xenografts.

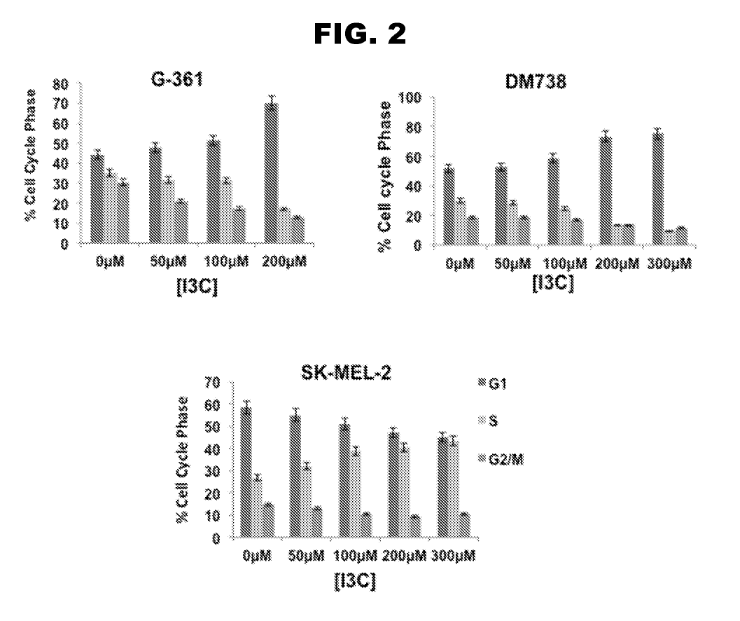

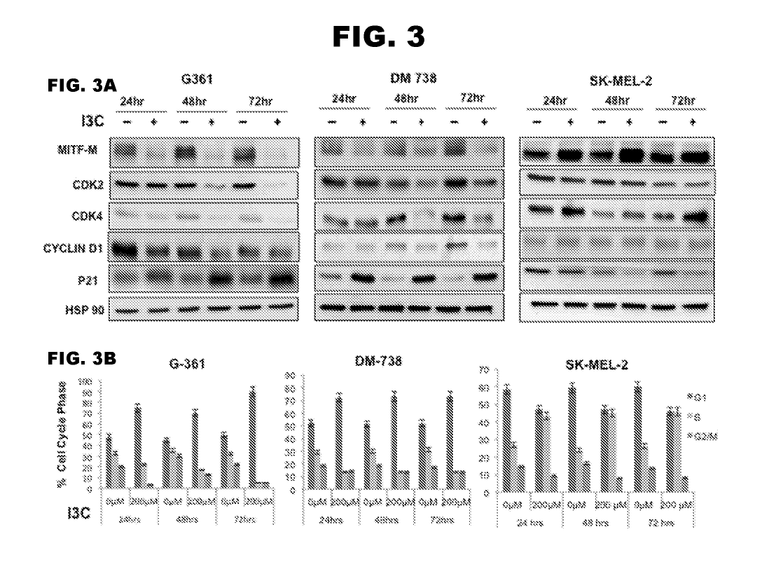

The potential effects of I3C on melanoma cell proliferation and production of MITF-M was tested in five different human melanoma cell lines, with distinct mutagenic profiles including G-361 cells. Each of the cell lines was treated for 72 hours with or without 200 μM I3C, and cell proliferation determined using a CCK-8 proliferation assay. Most sensitive to the I3C anti-proliferative effects were G-361, DM738 and RPMI-7951 cells, which harbor the BRAF-V600E oncogenic mutation, although each cell line differs with respect to their NRAS, PTEN and P53 genotypes (FIG. 1C). Western blot analysis of melanoma cells treated with varying concentrations of I3C for 48 hours showed that I3C strongly down-regulated MITF-M levels in G-361 and DM738 cells at approximately the same concentration range (FIG. 1D, upper and middle set of panels). Consistent with the cell cycle regulators CDK2, CDK4, cyclin D1 and p21 being transcriptional target genes of MITF-M, the I3C inhibition of MITF-M production dose-dependently and temporally correlated with the G1 cell cycle arrest and down-regulated levels of cell cycle genes in BRAF-V600E expressing melanoma cells (FIG. 2, FIG. 3). In contrast, wild type BRAF expressing melanoma cell lines, SK-MEL-24 and SK-MEL-2, were significantly less sensitive to the anti-proliferative effects of I3C (FIG. 1C), SK-MEL-2 expressed high levels of MITF-M protein at all concentrations of I3C (FIG. 1D, lower set of panels) and did not down regulate MITF-M targets or display a G1 cell cycle arrest in the presence of I3C (FIG. 2, FIG. 3).

FIG. 1: Effects of I3C on in vivo growth of melanoma tumor xenografts, production of MITF-M, and melanoma cell proliferation. (FIG. 1A) Athymic mice with G-361 cell-derived tumor xenografts were injected subcutaneously with either I3C or with DMSO vehicle control, and resulting tumor volumes were calculated as described. The micrograph insert shows tumors harvested at week 4. (FIG. 1B) At terminal sacrifice, tumor sections were analyzed for MITF-M expression by immunofluorescence using primary antibodies to MITF-M (left panel). Cultured G361 cells treated with I3C for 72 hr were similarly probed for MITF-M levels (Right panel). (FIG. 1C) Human melanoma cell lines with distinct genotypes were treated with or without 200 μM I3C for 48 hours and the effects on cell proliferation measured using a CCK-8 assay relative to the vehicle control. (FIG. 1D) The levels of MITF-M protein were determined in melanoma cells treated with the indicated concentrations of I3C for 48 hours by western blots.

FIG. 2: Dose dependent cell cycle effects of I3C in G361, DM738 and SK-MEL-2 melanoma cells. Human melanoma cells expressing either BRAF-V600E (G361 and DM738) or wild type BRAF (SK-MEL-2) were treated with the indicated concentration of I3C and harvested cells were stained with a hypotonic solution containing propidium iodide, and the DNA content of stained nuclei were quantified as described. The histograms of representative experiments from three independent experiments are shown and the percentage of cells in the population displaying G1, S or G2/M DNA content was quantified. As shown in this figure, I3C induced a significant G1 cell cycle arrest of BRAF-V600E expressing G361 and DM738 cells at the maximal dose of 200 μM I3C. In contrast, wild type BRAF expressing SK-MEL-2 cells remained in a rapidly proliferating state at all tested concentrations of I3C.

FIG. 3: Time course of the cell cycle effects of I3C in human melanoma cells expressing BRAF-V600E or wild type BRAF. Cultured human melanoma cell lines that express either an oncogenic mutant BRAF (G361 or DM738) or wild type BRAF (SK-MEL-2) were treated with or without 200 μM I3C over a 72 hour time course. (A) Total cell lysates were fractionated by SDS-polyacrylamide electrophoresis and the levels MITF-M, and CDK2, CDK4, Cyclin D1, p21 were determined by western blot analysis in comparison to the HSP90 gel loading control. Representative blots from three independent experiments are shown. (B) Cells were stained with a hypotonic solution containing propidium iodide, and the DNA content of stained nuclei were quantified by flow cytometric analysis as described in the methods and materials section. Representative histograms from three independent experiments are shown and the percentage of cells in the overall population displaying G1, S or G2/M DNA content was quantified.

### Example 2: Expression of Exogenous MITF-M Rescues the I3C Down-Regulation of the MITF-M Target Genes CDK2 and CDK4 and Attenuates the I3C Proliferative Arrest of Melanoma Cells

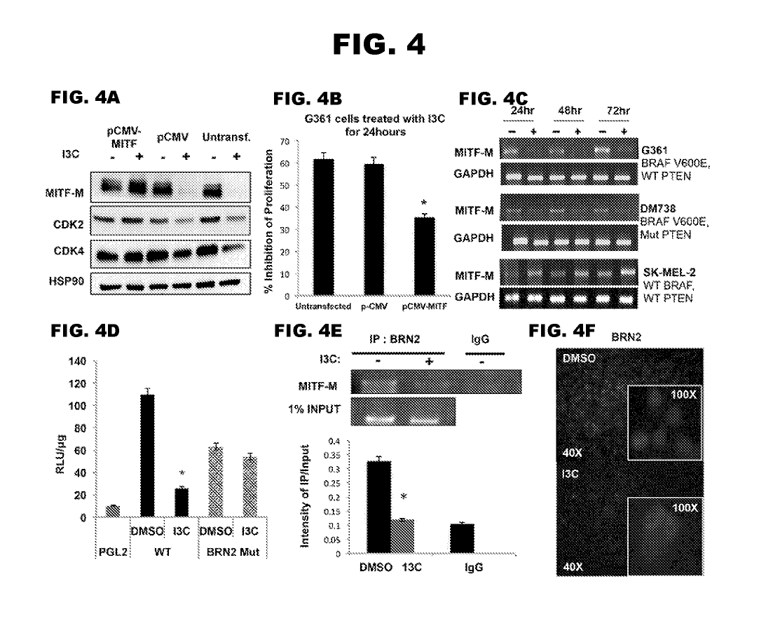

Exogenous MITF-M was expressed in G-361 melanoma cells to functionally test whether the I3C induced down regulation of MITF-M is required to mediate the I3C anti-proliferative response. Cells transfected with an MITF-M expression vector (pCMV-MITF) displayed high levels of MITF-M protein in the presence or absence of I3C, whereas, MITF-M protein levels were strongly down regulated by I3C in empty expression vector (pCMV) transfected cells or in untransfected cells (FIG. 4A). Western blots further revealed that the I3C down-regulation of two MITF-M target genes, CDK2 and CDK4, was rescued in cells expressing exogenous MITF-M protein (FIG. 4A). Consistent with a critical role of MITF-M down-regulation for the I3C anti-proliferative effects, expression of exogenous MITF-M protein strongly attenuated this process in cells treated with I3C for 24 hours (FIG. 4B). Empty vector transfected or untransfected melanoma cells showed only a 60% proliferative arrest because of the submaximal incubation time of 24 hours employed to ensure maximal expression of exogenous MITF-M.

FIG. 4: Role of MITF-M in I3C anti-proliferative response and I3C regulation of MITF-M gene expression. (FIG. 4A) G-361 cells were either transfected with pCMV-MITF expression vector, or pCMV empty vector control or left untransfected, and each set of cells were treated with or without 200 μM I3C for a submaximal time of 24 hours. Levels of MITF-M, CDK2, CDK4 and HSP90 protein were determined by western blots (Left panel). (FIG. 4B) Cell proliferation was measured using a CCK-8 assay, and results show the mean of three independent experiments±SEM (*, p<0.01). (FIG. 4C) MITF-M transcript expression in G-361 cells treated with or without 200 μM I3C was determined by RT-PCR analysis in comparison to the GAPDH control. (FIG. 4D) Cells were transfected with reporter plasmids containing either a wild type MITF-M promoter (WT), a Brn2 consensus site mutant (Brn2 Mut) or the PGL2 empty control vector. Luciferase specific activity was measured in cells treated with or without 200 μM I3C for 24 hours. The bar graph shows the results of three independent experiments in triplicate±SEM (*, p<0.01). (FIG. 4E) ChIP assay was performed on G361 cells treated with or without 200 μM I3C for 48 hours using BRN2 antibodies (IP:BRN2) or the control IgG with one percent input as the loading control. The bar graphs quantify the densitometry results from three independent experiments±SEM (*, p<0.01). (FIG. 4F) G-361 cells were treated with or without 200 μM I3C for 48 hours and BRN2 localization was examined by immunofluorescence using anti-BRN2 antibodies. The inserts are magnified portions of the larger fields.

### Example 3: I3C Disrupts BRN2 Interactions with the MITF-M Promoter Inhibiting Promoter Activity and Gene Expression

I3C effects on MITF-M transcript levels were examined in BRAF-V600E expressing G-361 and DM738 cells as well as wild type BRAF-expressing SK-MEL-2 cells treated for a 72 hour time course with or without 200 μM I3C. RT-PCR analysis of total RNA at each time point revealed that I3C strongly down regulated MITF-M transcript levels in both the BRAF-V600E expressing melanoma cell lines, accounting for the loss of MITF-M protein (FIG. 4C, upper and middle panels). G-361 cells displayed a greater loss of MITF-M transcripts compared to DM738 cells. In contrast, in wild type BRAF-expressing SK-MEL-2 cells, I3C induced a modest increase in MITF-M transcript levels (FIG. 4C, lower panels). Consistent with the loss of MITF-M transcripts, transient transfection of a −333/+120 MITF-M promoter-luciferase reporter plasmid (WT) revealed that I3C strongly down regulated MITF-M promoter activity (FIG. 4D). This MITF-M promoter fragment contains a consensus site for the melanoma specific transcription regulator BRN2 (N-Oct3) at −50 to −36, which functions downstream of the oncogenic BRAF signaling pathway to maintains high levels of melanoma MITF-M (Wellbrock and Arozarena, Pigment Cell Melanoma Res. 2015. 28:390-406). Mutation of the Brn2 consensus site (BRN2 Mut), prevented the I3C down regulation of MITF-M promoter activity (FIG. 4D), directly implicating involvement of BRN2 in the I3C inhibition of MITF-M promoter activity.

Chromatin immunoprecipitation (ChIP) assay demonstrated that I3C treatment disrupted binding of endogenous BRN2 to the MITF-M promoter. G-361 cells were treated with or without I3C for 48 hours, and the genomic fragments cross-linked to protein were immunoprecipitated with anti-BRN2 antibodies or with an IgG control antibody. PCR analysis using primers specific to the BRN2 binding site in the MITF-M promoter revealed that I3C significantly down regulated endogenous BRN-2 interactions with this promoter (FIG. 4E). One percent input was used as a loading control. Immunofluorescence of G-361 melanoma cells treated with or without 200 μM I3C for 48 hours revealed that I3C disrupted the nuclear localization of Brn2 and thereby preventing accessibility of this transcription factor to the MITF-M promoter. In vehicle control DMSO treated cells, BRN2 is highly concentrated in the nucleus (FIG. 4F, upper panel), whereas, treatment with I3C disrupted the nuclear localization of BRN2 with this protein remaining diffusely localized throughout the cytoplasm (FIG. 4F, lower panel).

### Example 4: I3C Disrupts the Oncogenic BRAF Signaling Pathway

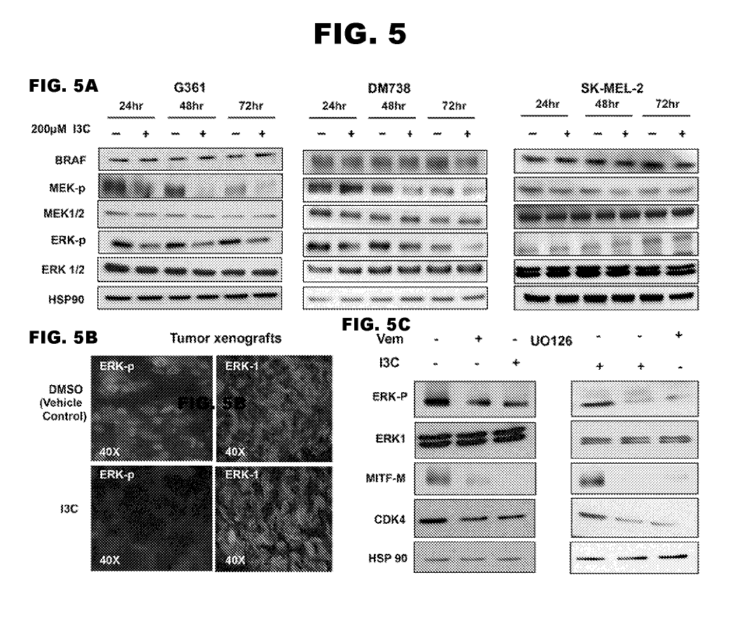

To assess the potential effects of I3C on the immediate downstream effectors of BRAF signaling, namely MEK and ERK/MAPK, melanoma cells were treated with or without I3C over a 72 hour time course and protein levels of each signal transduction component was examined by western blots. In the BRAF-V600E expressing G-361 and DM738 cells, I3C strongly down regulated the active phosphorylated forms of MEK and Erk/MAPK with no corresponding changes in the level of total MEK or Erk/MAPK protein respectively (FIG. 5A, Left and Middle panels). The total levels of BRAF-V600E protein and the control protein Hsp90 remained unchanged. In contrast, I3C had no effect on the levels of phosphorylated MEK or phosphorylated Erk/MAPK in wild type BRAF expressing SK-MEL-2 cells (FIG. 5A Right panel), which likely accounts for the inability of I3C to down-regulate MITF-M expression in this melanoma cell line (FIG. 1D). The in vivo effect of I3C on oncogenic BRAF signaling was analyzed by immunofluorescence on sections from G-361 cell-derived tumor xenografts. Consistent with the cellular observations, I3C strongly down regulated the level of phosphorylated Erk/MAPK with no effect on the total levels of Erk/MAPK protein in the tumor xenografts (FIG. 5B).

In a complementary pharmacological approach, G-361 cells were treated for 48 hours with or without either 10 μM Vemurafenib, a clinically used BRAF-V600E specific inhibitor (Flaherty et al., Nat. Rev. Drug Discovery 2011. 10:811-812), or 10 μM UO126, a selective inhibitor of MEK1/2 activity (Shi et al., Pharmazie 2014. 69:346-352). A parallel set of cells were treated with 200 μM I3C and the effects compared to each inhibitor. I3C, Vemurafenib and U0126 each down regulated the levels of phosphorylated Erk/MAPK, MITF-M and CDK4 protein (FIG. 5C). The Vemurafenib and U0126 effects on MITF-M protein in melanoma cells is the first such observation for either pharmacological inhibitor. Taken together, these results indicate that in melanoma cells I3C disrupts oncogenic BRAF signaling at or upstream of MEK activation.

FIG. 5: Effect of I3C on BRAF signalling in cells and in vivo tumors. (FIG. 5A) BRAF-V600E expressing G-361 and DM-738 cells as well as wild type BRAF expressing SK-MEL-2 cells were treated with or without 200 μM I3C for 24, 48 and 72 hours. Western blots were performed on total cell extracts and probed with the indicated antibodies. (FIG. 5B) Tumor xenograft sections from I3C treated and untreated animals were analyzed for ERK-p and total ERK-1 protein by immunofluorescence. (FIG. 5C) G-361 cells were treated with or without 10 μM Vemurafenib BRAF inhibitor, 10 μM U0126 MEK inhibitor or 200 μM I3C for 48 hours. Western blots were probed for the indicated proteins and the results are representative of three independent experiments.

### Example 5: In Silico Modeling Predicts a Stable I3C Binding Site Specific to the Oncogenic BRAF-V600E

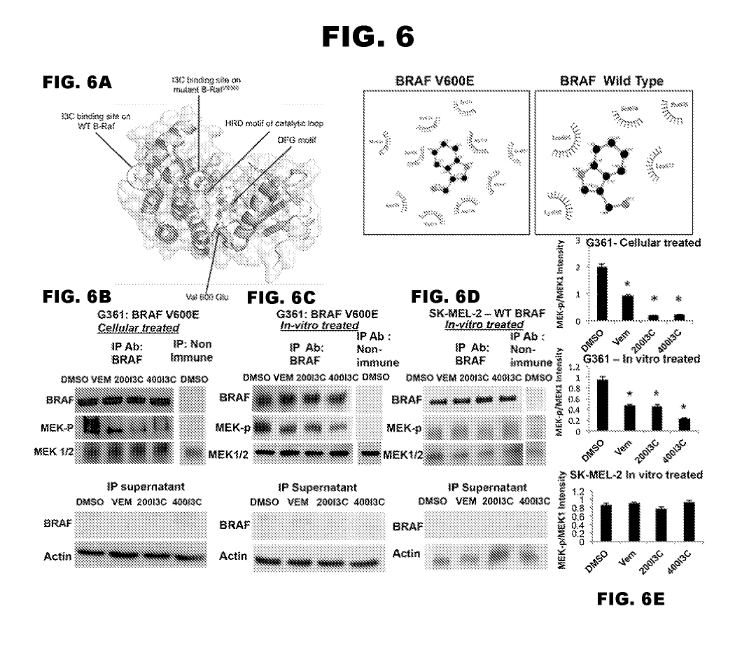

To initially test the possibility that I3C directly interacts with BRAF, computer-aided binding simulations were performed that utilize the known 3-D crystal structures of the mutant and wild type BRAF proteins and the chemical structure of I3C (FIG. 6A). Using the Hex Protein Docking software to scan the BRAF protein surfaces for potential docking sites with I3C, the molecular dynamics and thermo-stability are calculated using free energy simulations. Inspection of calculated interactions of I3C with BRAF-V600E using the LigPlot program predicts that I3C makes Van der Waals interactions (within 3.5 Å) with two critical catalytic residues Arg 575 and Asp 576 that are located in the HRD motif of the catalytic loop of BRAF-V600E. I3C is also predicted to form Van der Waals interactions with Met 620, Val 624, Ser 616, Tyr 633, Leu 577, Ala 641, Tyr 619, and Lys 578 that are generally within the catalytic loop (FIG. 6A). Interestingly, the predicted I3C interacting residues in BRAF-V600E are in close proximity (less than 10 Å) to the site of the oncogenic mutation at position 600, which could explain the selectivity of I3C for the mutant BRAF protein. In contrast, simulations that used the wild type BRAF protein structure predict that I3C binds at the surface of the protein by its potential interactions with amino acid residues Leu 696, Lys 697, Ser 674, Pro 675, Leu 677), which are all located away from critical domains involved in catalysis (FIG. 6A).

FIG. 6: In silico and in vitro demonstration that I3C is an inhibitor of oncogenic BRAF-V600E enzymatic activity. (FIG. 6A) Predictive binding simulations of I3C interactions with mutant BRAF-V600E and wild type BRAF were analyzed using the PyMol program (left side simulation). The LigPlot program examined Van der Waals interactions (within 3.5 Å) between I3C and amino acids in either BRAF-V600E or wild type BRAF (right side simulation). (FIG. 6B) G-361 cells were treated with 200 μM I3C, 400 μM I3C, 10 μM Vemurafenib or with DMSO vehicle control for 48 hours. BRAF was immunoprecipitated from pretreated cell extracts, and assayed for its intrinsic enzymatic activity in vitro using inactive MEK as the substrate in the presence of ATP. Immunoprecipitation with non-immune IgG was used as a negative control. The level of in vitro generated phospho-MEK was determined by western blot analysis (left panel). The lower panels show the level of BRAF remaining in the cell extracts after immunoprecipitation by western blot analysis. (FIG. 6C) BRAF-V600E was immunoprecipitated from untreated G-361 cells and incubated in vitro with 200 μM I3C, 400 μM I3C, 10 μM Vemurafenib or the DMSO vehicle control. BRAF-V600E enzymatic activity was assayed using the level of detected in vitro phosphorylation of inactive MEK as described above. (FIG. 6D) Wild type BRAF was immunoprecipitated from SK-MEL-2 melanoma cells and BRAF enzymatic activity post I3C treatment was analyzed as described for FIG. 4C. (FIG. 6E) I3C regulation of BRAF enzymatic activity in vitro was quantified by densitometry of MEK-P and total MEK protein levels detected by western blots from three independent experiments±SEM (*, p<0.01).

### Example 6: I3C Directly Inhibits Catalytic Activity of the Oncogenic BRAF-V600E but not the Wild Type BRAF

The predicted I3C interactions with essential catalytic residues in the mutant BRAF-V600E protein, in particular Arg 575 and Asp 576, suggest that I3C may selectively disrupt BRAF-V600E enzymatic activity. To initially test whether I3C alters BRAF-V600E activity in cells, G-361 cells were treated for 48 hours with the DMSO vehicle control, 200 μM I3C, 400 μM I3C or with 10 μM Vemurafenib. The BRAF-V600E protein was pulled down from each of the cell extracts with anti-BRAF antibodies and the kinase activities assayed in vitro by adding ATP and inactive MEK protein as the BRAF substrate. After 30 minutes incubation, the levels of phosphorylated MEK protein in the reaction mixture was assessed using western blots to measure of BRAF-V600E activity. Cellular treatment with either I3C or Vemurafenib significantly inhibited BRAF-V600E enzymatic activity as shown by the loss of phosphorylated MEK, whereas, in the absence of either compound, immunoprecipitated BRAF-V600E was highly active (FIG. 6B, IP:BRAF panels). The levels of BRAF-V600E, MEK and the Hsp90 gel loading control in each of the reaction mixtures remained constant.

The in vitro kinase assay was further used to assess the potential direct effects of I3C on BRAF enzymatic activity. BRAFV600E or wild type BRAF protein were first immunoprecipitated from untreated G-361 or SK-MEL-2 melanoma cells respectively and then pre-incubated for 2 hours with the DMSO vehicle control, 200 μM I3C, 400 μM I3C or 10 μM Vemurafenib. Subsequently inactive MEK protein and ATP were added to in vitro reactions and after 30 min the level of BRAF activity was assessed by western blots to determine the level of phosphorylated MEK protein. I3C strongly down regulated BRAF-V600E activity at levels comparable to the well-characterized BRAF-V600E inhibitor Vemurafenib (FIG. 6C, left panels). In contrast, in vitro incubations containing either I3C or Vemurafenib had no effect on wild type BRAF enzymatic activity (FIG. 6D, right panels). The relative BRAF activities generated under each condition were quantified by densitometry of the amount of phosphorylated MEK detected in the western blots (FIG. 6E). The observed BRAF-V600E kinase activity was dependent on the use of anti-BRAF antibodies in the original immune isolations because no phosphorylated MEK was observed when non-immune antibodies were used for the immunoprecipitations (FIG. 6B, FIG. 6C and FIG. 6D, upper panels). Furthermore, the post-pull down supernatant fractions were devoid of any residual BRAF protein showing that the BRAF immunoprecipitations quantitatively brought down all of the BRAF protein in each cell extract (FIG. 6B, FIG. 6C and FIG. 6D, lower panels). Taken together, these results demonstrate that I3C functions in human melanoma cells as a direct and selective inhibitor of BRAF-V600E enzymatic activity.

### Example 7: Anti-Proliferative Effects of Combinations of I3C and Vemurafenib in Oncogenic BRAF-V600E Expressing Melanoma Cells

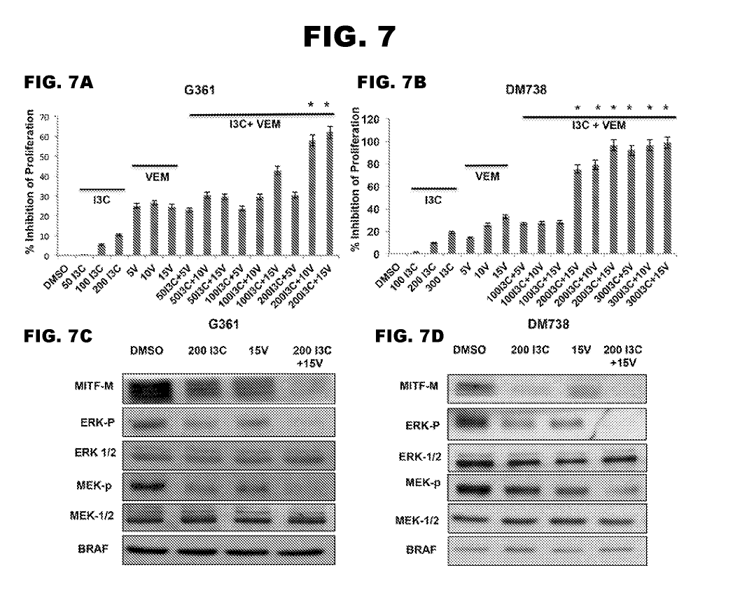

Given that I3C and Vemurafenib have different binding sites on the oncogenic BRAF, it was investigated whether a combination of I3C and Vemurafenib could induce a stronger anti-proliferative response in sensitive melanoma cells compared to each compound alone. BRAF-V600E expressing G-361 and DM738 melanoma cells were treated with different concentration combinations of I3C and Vemurafenib for a submaximal response time of 24 hours and cell proliferation was determined by CCK-8 assay. A combination of I3C and Vemurafenib more effectively inhibited the proliferation of both melanoma cell lines compared to the effects of either I3C or Vemurafenib alone (FIG. 7A-7B). The effects of 24 hour treatments with 200 μM I3C and/or 15 μM Vemurafenib on BRAF-V600E signaling was examined in G-361 and DM738 melanoma cell. Western blot analysis of total cell extracts revealed that the I3C and Vemurafenib combination down-regulated MITF-M, phosphorylated Erk/MAPK and phosphorylated MEK to a significantly greater extent compared to the individual effects of either I3C or Vemurafenib (FIG. 7C-7D). The down regulation of MITF-M protein levels mirrored the arrest of melanoma cell proliferation.

FIG. 7: Effects of combinations of I3C and Vemurafenib on melanoma cell proliferation and BRAF-V600E signaling. G-361 (FIG. 7A) or DM-738 cells (FIG. 7B) were treated with the indicated combinations of I3C and Vemurafenib for a suboptimal time of 24 hours and inhibition of proliferation was monitored using a CCK-8 assay. The results represent the average of three independent experiments with a mean±SEM (*, p<0.01) shown in the bar graphs. G-361 cells (FIG. 7C) or DM-738 cells (FIG. 7D) were treated with combinations of 200 μM I3C and/or 15 μM Vemurafenib for 24 hours and the levels of the indicated proteins determined by western blots on total cell extracts.

### Example 8: 1-Benzyl I3C Causes Antiproliferation in Melanoma Cell Culture and In-Vivo at >10-Fold Lower Concentration than I3C and Shows Sensitivity Over a Wider Range of Mutation Profiles

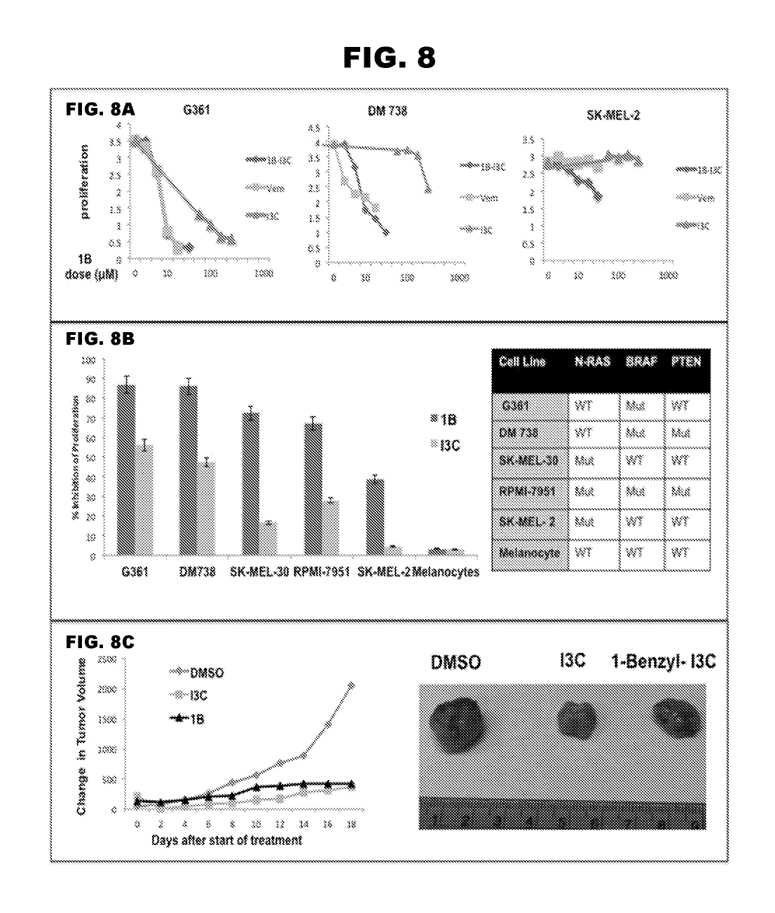

To test if 1-benzyl I3C showed antiproliferative effects in melanoma comparable to its parent compound I3C, two I3C sensitive cell lines, G-361 and DM 738 were treated with varying doses of 1-benzyl I3C for 48 hours and cell proliferation was determined using CCK-8 assay. 1-benzyl I3C not only showed significantly greater antiproliferation than I3C but also at 10-20 μM concentration that is 10-20 fold lower than the optimum concentrations of I3C at 200-300 μM. Further, these concentrations of 1-benzyl I3C were similar to the optimum concentrations of the clinically used BRAF inhibitor Vemurafenib. Interestingly, unlike I3C and Vemurafenib, which are ineffective in melanoma cell lines expressing wild-type BRAF such as SK-MEL-2, 1-benzyl I3C showed antiproliferation in these cells as well. This indicated that 1-benzyl I3C possibly has a different mechanism of action that was independent of the presence of oncogenic BRAF (FIG. 8A).

To test if there was any mutational determinant to predict sensitivity to 1-benzyl I3C, 5 different melanoma cells lines with mutation profiles encompassing some of the most common mutations associated with melanoma namely BRAF, PTEN, N-RAS and P53 were treated with the optimum dose of 20 μM 1-benzyl I3C and 200 μM I3C for 48 hours. 1-benzyl I3C showed a much wider range of sensitivity than I3C and Vemurafenib being effective in cell lines harboring wild-type BRAF as well (FIG. 8B). No such antiproliferative response was observed upon treatment of normal epidermal melanocytes with 1-benzyl I3C indicating that it specifically targets melanoma cells and therefore has translatable potential.

This study was extended to an in vivo murine model of melanoma generated by injecting G-361 cells into the flanks of athymic nude immunocompromised mice to generate palpable xenografted tumors. The mice were subcutaneously injected with the optimum dose of 20 mg/kg body weight 1-benzyl I3C everyday, for a 4-week time course and the size of the tumors were measured every other day and volumes calculated as described in the methods. The treatment could be extended beyond a month since the vehicle treated tumor size was beyond the permitted size for humane animal treatment for research. 1-benzyl I3C strongly attenuated tumor growth compared to the vehicle control that was noticeable within the first week of injections and sustained over the entire time course (FIG. 8C). At termination of treatment, the mice were sacrificed and the residual tumors were harvested. The size of the residual tumors treated with 1-benzyl I3C was significantly smaller than the vehicle control and comparable to the tumors treated with 10 fold higher concentration of I3C (FIG. 8C, right panel). This confirmed that like in breast cancer, 1-benzyl I3C is a significantly more potent molecule than I3C even against melanoma.

FIG. 8: Potency of 1-benzyl I3C against human melanoma in cell culture and in vivo melanoma tumor xenografts. (FIG. 8A) Oncogenic BRAF expressing and I3C, Vemurafenib sensitive G361 and DM738 cells as well as Wild type BRAF expressing and I3C, Vemurafenib resistant SK-MEL-2 cells were treated with 20 μM 1-benzyl I3C. Its effect on proliferation of these cells was determined using a CCK-8 cell proliferation assay. (FIG. 8B) Human melanoma cell lines with distinct genotypes were treated with or without 20 μM 1-benzyl I3C for 48 hours and the effects on cell proliferation measured using a CCK-8 assay relative to the vehicle control. (FIG. 8C) Immunocompromised athymic nude mice with G-361 cell-derived xenografted tumors were subcutaneously injected with either 1-benzyl I3C or with DMSO vehicle control, and resulting tumor volumes were calculated as described in the Material and Method section. The micrograph insert shows tumors harvested at week 4.

### Example 9: 1-Benzyl I3C Downregulates Key Cell-Cycle Regulators to Cause a G1 Phase Arrest in Sensitive Cells

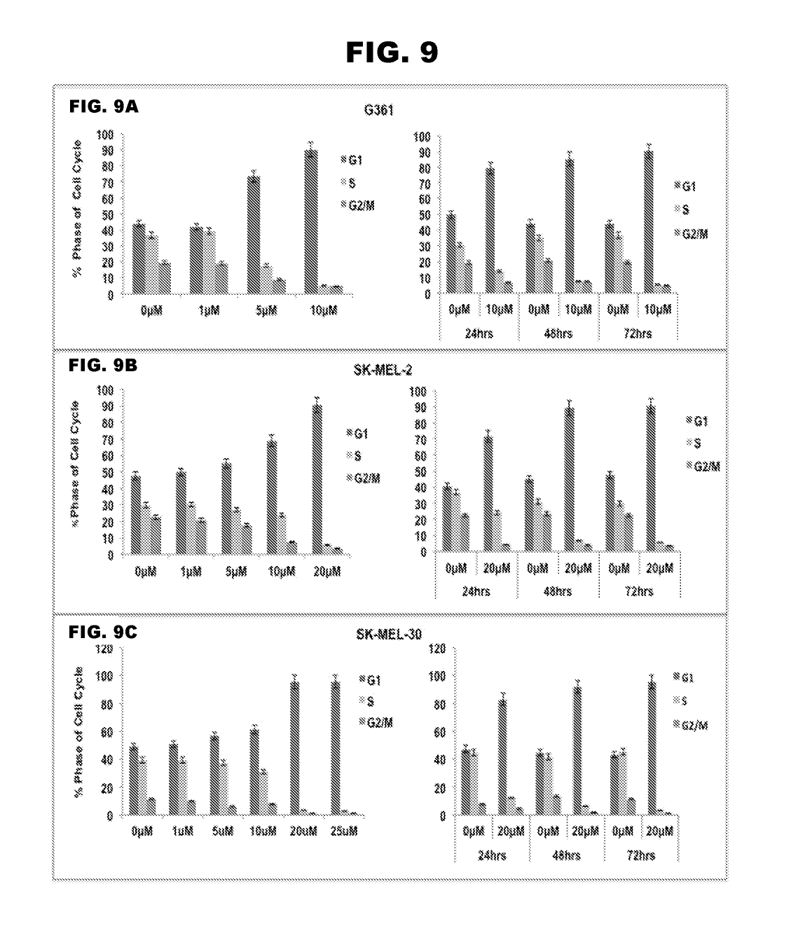

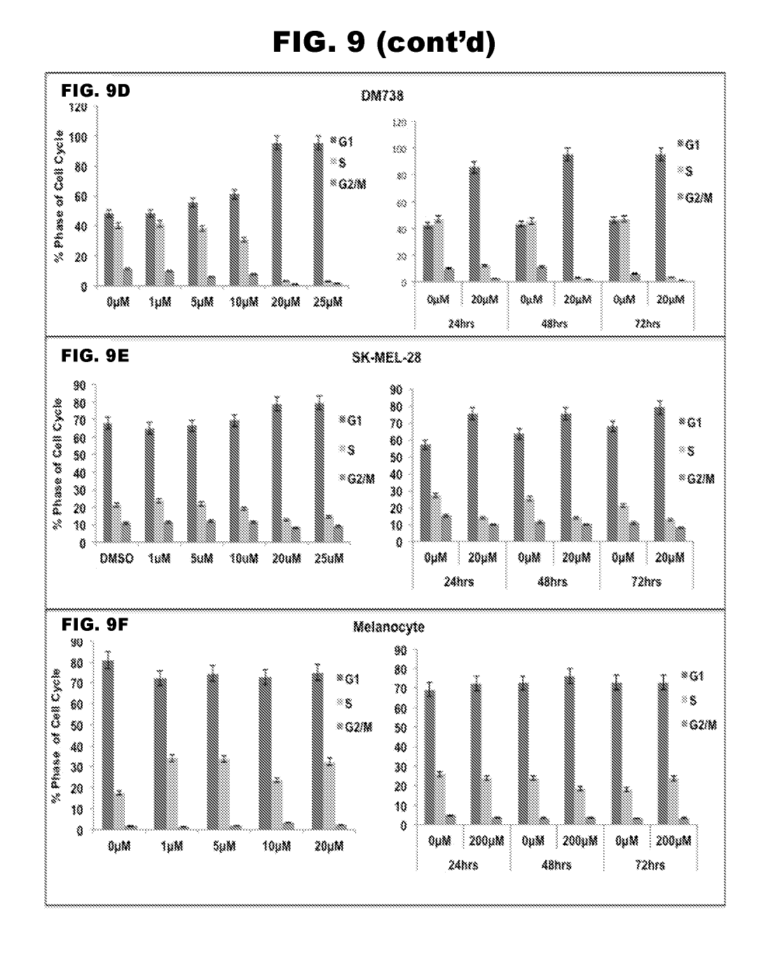

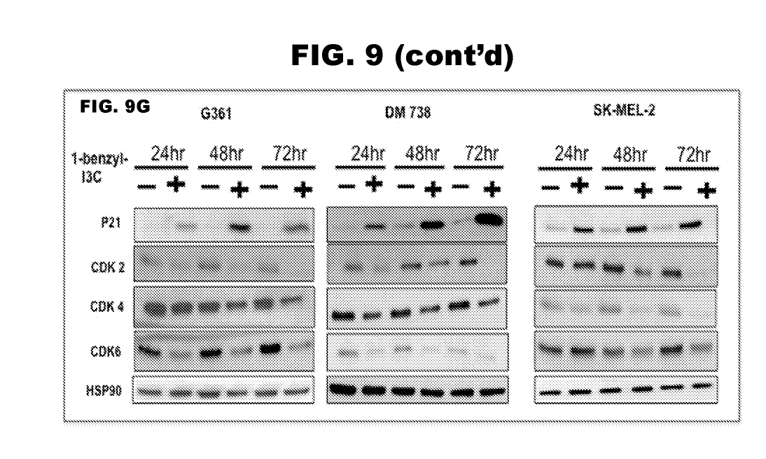

To understand the molecular mechanism underlying this antitumor effect, flow cytometric analysis was performed on multiple melanoma cell lines as well as normal melanocytes, treated with varying doses of 1-benzyl Indole-3-carbinol for 48 hours. A significant G1 cell cycle arrest was observed in all the melanoma cell lines with no comparable effect on normal melanocytes, implying that 1-benzyl Indole-3-carbinol selectively affects melanoma cells by inducing a G1 cell cycle arrest (FIG. 9A-9F, left panel). Next, similar flow cytometric analysis was performed on the cell lines with the optimum dose of 20 μM 1-benzyl I3C for a time course of 24, 48, 72 hours to examine the temporal profile of its antiproliferative effect (FIG. 9A-9F, right panel). Western blot analyses performed on cell lines treated with 1-benzyl I3C for 24, 48, 72 hours revealed downregulation of key cell cycle regulators like CDK 2, 4, 6, concomitant to the G1 cell cycle arrest induced by 1-benzyl Indole-3-carbinol (FIG. 9G).

FIG. 9: Dose dependent and temporal effects of 1-benzyl I3C on the cell cycle in melanoma cell lines with a range of mutation profiles. Human melanoma cells namely G361, DM738 and SK-MEL28 (FIG. 9A, 9D, 9E) expressing oncogenic BRAF-V600E and wild type BRAF expressing SK-MEL-2 and SK-MEL-30 (FIG. 9B, 9C) cells as well as normal epidermal melanocytes were treated with the indicated concentrations of 1-benzyl I3C for indicated time durations. Harvested cells were stained with propidium iodide, and the DNA content of stained nuclei were quantified by flow cytometric analysis as described in the Materials and Method section. The histograms of representative experiments from three independent trials are shown and the percentage of cells in the population displaying G1, S or G2/M DNA content was quantified. In FIG. 9 the Left panels show that 1-benzyl I3C induced a dose-dependent G1 cell cycle arrest in all of the cell lines examined and the response, unlike I3C was independent of presence of oncogenic BRAF-V600E or PTEN. The right panel shows the 24, 48 and 72 hours treatment response profile of the same cell lines. Evidently in all the cell lines 1-benzyl I3C induces a significant G1 cell cycle arrest as early as within 24 hours of treatment and the effect persists through 48 to 72 hours. In case of cell lines like G361, SK-MEL-2 and SK-MEL-30, the G1 arrest accentuates with time. Additionally 1-benzyl I3C did not show any such G1 phase cell cycle arrest in the normal epidermal melanocytes accounting for the resistance of these cells to the anti-proliferative effect of 1-benzyl I3C as observed in the CCK-8 proliferation assay. (FIG. 9G) Effects of 1-benzyl I3C treatment on key cell cycle regulators over a time course in human melanoma cells. Cultured human melanoma cell lines that express oncogenic mutant BRAF V600E alongside wild type PTEN (G361) or mutant PTEN (DM738) or wild type BRAF and wild type PTEN expressing (SK-MEL-2) cells were treated with or without 20 μM I3C over a 72 hours time course. Total cell lysates were fractionated by SDS-polyacrylamide electrophoresis and the levels cell cycle regulators like CDK2, CDK4, Cyclin D1 and p21 were determined by western blot analysis in comparison to the HSP90 gel loading control. Representative blots from three independent experiments are shown.

### Example 10: Inhibition of MITF-M Expression by 1-Benzyl I3C is Necessary for Induction of its Antiproliferative Effect

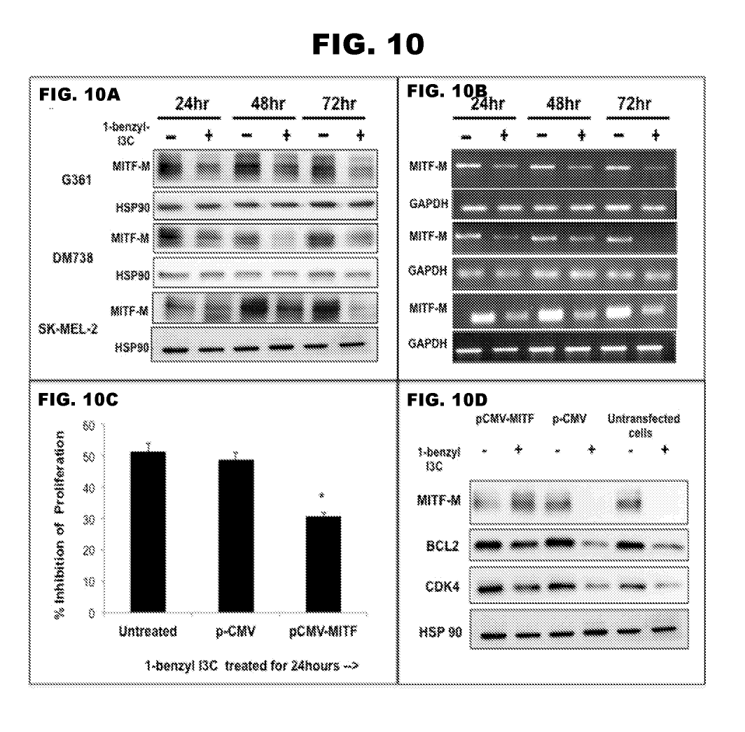

Since the 1-benzyl I3C inhibited cell cycle regulators are downstream targets of the melanoma master regulator—MITF-M, it was logical to examine if it was affected by 1-benzyl I3C treatment as well. Western blots on G361 and DM738 cells treated with 1-benzyl I3C for 24, 48, 72 hours showed a significant downregulation of MITF-M starting as early as 24 hours similar to observations with I3C treatment. Interestingly, the WT BRAF expressing SK-MEL-2 cells which were not sensitive to I3C or Vemurafenib and showed no downregulation of MITF-M levels upon treatment with I3C, shows a significant inhibitory effect on MITF-M protein levels with 1-benzyl I3C treatment (FIG. 10A). RT PCR performed on these cells post 1-benzyl I3C treatment showed a concomitant inhibition of MITF-M transcript level accounting for the observed downregulation of MITF-M protein (FIG. 10B).

To further determine if MITF-M is necessary to mediate 1-benzyl Indole-3-carbinol's antiproliferative effect, MITF-M was overexpressed in sensitive G361 cells by transiently transfecting it with plasmid expressing MITF-M (pCMV-MITF). Subsequently, the cells were treated with or without 1-benzyl I3C for 24 hours and proliferation was determined using a CCK-8 assay. Overexpressing MITF-M not only rescued 1-benzyl Indole-3-carbinol's antiproliferative effect, but also the downregulation on cell cycle regulator genes like CDK4 as well as pro-apoptotic genes like BCL2 as determined by western blots (FIG. 10C-10D).

FIG. 10: 1-benzyl I3C regulation of MITF-M expression to mediate its anti-proliferative effect in melanoma. (FIG. 10A) The levels of MITF-M protein were determined in melanoma cells treated with 10 μM concentration of 1-benzyl I3C for a time course of 24, 48 and 72 hours by western blots. (FIG. 10B) The effect of 1-benzyl I3C on MITF-M transcript expression in the same cells as FIG. 3A namely G-361, DM738, SK-MEL-2, treated with or without 20 μM I3C was determined by RT-PCR analysis in comparison to the GAPDH control. (FIG. 10C) G-361 cells were either transfected with pCMV-MITF expression vector, or pCMV empty vector control or left untransfected, and each set of cells were treated with or without 20 μM I3C for a submaximal time of 24 hours. Cell proliferation was measured using a CCK-8 assay, and results show the mean of three independent experiments±SEM (*, p<0.01). (FIG. 10D) Levels of MITF-M and its downstream targets—anti-apoptotic BCL2 and pro-proliferative CDK4 protein levels were determined in comparison to HSP90 by western blots.

### Example 11: 1-Benzyl I3C Disrupts Signaling Through the Wnt/β-Catenin Pathway

The molecular mechanism by which 1-benzyl Indole-3-carbinol downregulated levels of MITF-M transcripts was investigated. The observation that the antitumor effect of the compound is not restricted to the BRAF V600E expressing melanoma cell lines, led to the investigation of its effect on other signaling pathways known to regulate MITF-M. Since one-third of human melanomas specimens show activation of the canonical Wnt pathway evident from the presence of nuclear β-catenin, whether 1-benzyl I3C treatment affected the Wnt/β-catenin pathway was tested (Rohinton et al., Carcinogenesis 2010. 31(10):1844-1853). Additionally, recent studies have implicated β-catenin signaling in a key role in melanoma progression, tumor cell survival and chemoresistance (Sinnberg et al., PLoS ONE 2011. 6(8)). From these evidence it was hypothesized that a compound that downregulates β-catenin can potentially inhibit MITF-M expression to induce apoptosis in metastatic melanoma and will be effective against a wider range of melanoma phenotypes, since it would function independent of the presence of the oncogenic BRAF and 1-benzyl I3C seemed to fit the bill.

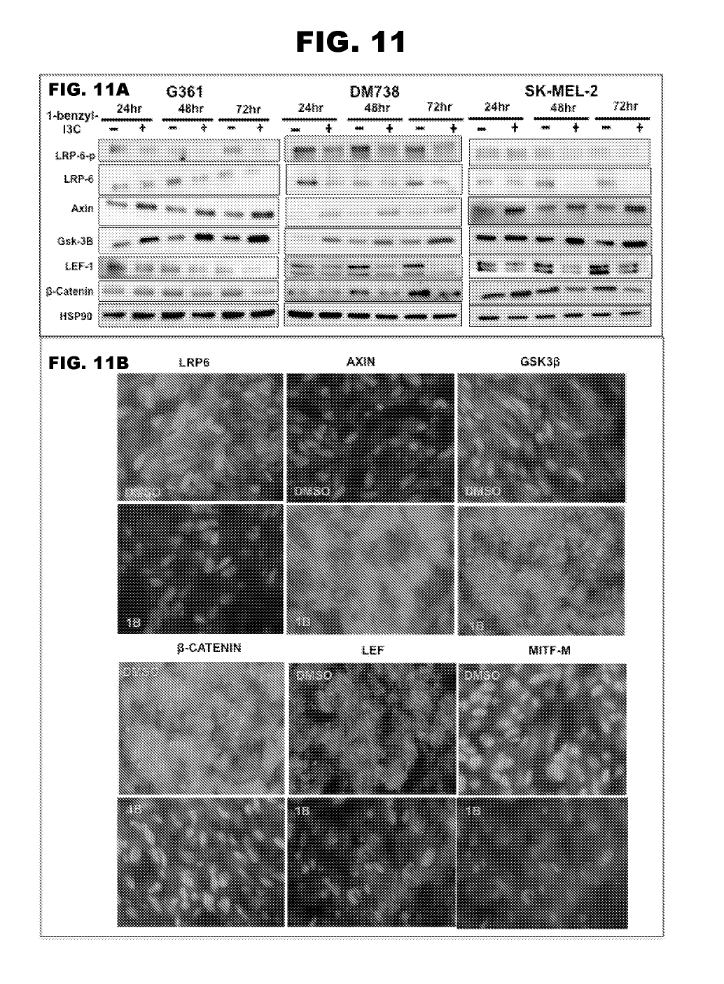

Western blots performed on melanoma cell lines treated with 1-benzyl I3C revealed that different components of the Wnt/β-catenin pathway were indeed significantly affected by the treatment. β-catenin protein levels were significantly downregulated in all the cell-lines, which could be explained by the observed upregulation of components of its destruction complex such as GSK3β and Axin, that function to restrict β-catenin to the cytoplasm and phosphorylates it for inactivation and subsequent degradation in absence of the growth factor Wnt. β-catenin therefore cannot enter the nucleus to perform its function of regulating the transcription of its target genes like MITF-M. Additionally the Wnt co-receptor LRP5/6 itself was significantly downregulated implicating that 1-benzyl I3C possibly binds directly to LRP5/6 or to some component of the Wnt signaling pathway functioning upstream of it (FIG. 11A). The effect of 1-benzyl I3C on the Wnt signaling pathway was further confirmed by immunofluorescence on sections taken from 1-benzyl I3C treated tumor xenografts, which mimicked the cell culture data, thereby reiterating the involvement of this signaling cascade in 1-benzyl I3C's anti-tumor effect (FIG. 11B).

FIG. 11: Effect of 1-benzyl I3C on Wnt signaling in cells and in vivo tumors. (FIG. 11A) BRAF-V600E expressing G-361 and DM-738 cells as well as wild type BRAF expressing SK-MEL-2 cells were treated with or without 20 μM 1-benzyl I3C for 24, 48 and 72 hours. Western blots were performed on total cell extracts and probed with the indicated antibodies related to the Wnt signaling pathway. (FIG. 11B) 10 micron cryostat sections of tumors harvested from 1-benzyl I3C and DMSO vehicle control treated animals were analyzed for the indicated players of the Wnt signaling pathway by immunofluorescence staining. The results are 63× magnified representative images from three independent experiments.

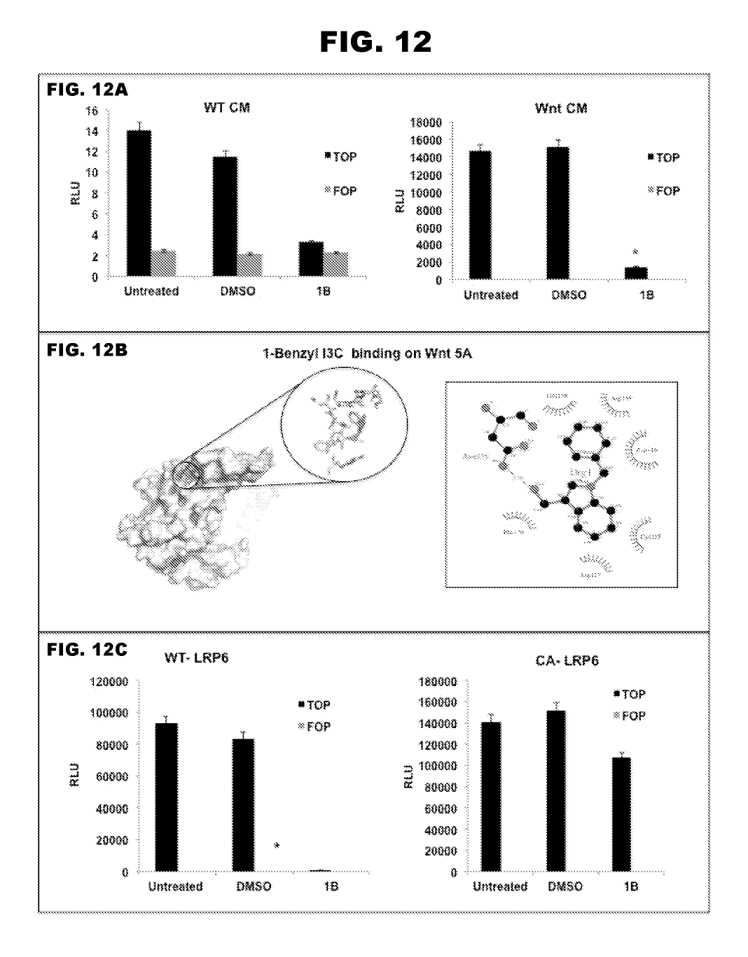

β-catenin functions at the node of multiple signal transduction pathways and is stabilized by Wnt independent mechanisms as well (Anastas et al., J. Clin. Invest. 2014. 124(7):2877-2890; Da Form et al., Clin. Cancer Res. Off. J. Am. Assoc. Cancer 2008. 14(18):5825-5832). Hence, to confirm the involvement of the canonical Wnt signaling in 1-benzyl I3C's mechanism of action, TOP FLASH assays were performed. G361 cells were cultured in conditioned media harvested from either mouse fibroblast cells (Lcells) stably transfected with empty CMV vector or L-cells expressing pCMV-Wnt. It is presumed that the conditioned media from the Wnt expressing cells will contain large amounts of the secreted growth factor and hence will be able to trigger Wnt signaling in the G361 cells. The G361 cells were transiently transfected with either a multiple LEF binding site containing enhancer driven minimal promoter luciferase reporter construct (TOP) or a similar construct in with mutant LEF binding site to serve a negative control (FOP). 24 hours post transfection, the TOP as well as FOP transfected cells were treated either with DMSO or 1-Benzyl-Indole-3-carbinol for 24 hours and the luciferase activity was measured. In the cells growing in the Wnt containing conditioned media, the DMSO vehicle control treated cells showed massive Wnt signaling detected by very high luciferase activity. Upon treatment with 1-benzyl Indole-3-carbinol, Wnt signaling was significantly attenuated evident from the loss of luciferase activity thereby confirming the involvement of the canonical Wnt signaling cascade in 1-benzyl Indole-3-carbinol's mechanism of action (FIG. 12A).

Using in-silico modeling to predict potential 1-benzyl Indole-3-carbinol binding site on different signaling component of the Wnt pathway indicated a potential direct binding site of our molecule on the growth factor Wnt itself (FIG. 12B) and not on its receptor Frizzled or the co-receptor LRP5/6. To indirectly test the veracity of this prediction TOP FLASH assays were performed like before but this time the cells were additionally co-transfected with either an empty CMV vector or a vector expressing constitutively active LRP6 (pCMV-LRP), which acts a co-receptor for Wnt with its direct binding receptor Frizzled. In the wild-type vector transfected cells, treatment with 1-benzyl I3C significantly inhibited the Wnt signaling compared to the vehicle treated control. In the constitutively active LRP6 transfected cells, 1-benzyl I3C induced inhibition of luciferase activity was significantly rescued (FIG. 12C). This shows that 1-benzyl I3C has a direct binding target upstream of LRP5/6, reiterating the possibility that 1-benzyl I3C could be directly binding to the Wnt ligand itself to disrupt its binding to Frizzled or disrupt formation of the receptor complex with Frizzled and LRP6, resulting in downregulation of the signaling cascade through GSK3β/β-Catenin/LEF/MITF-M.

FIG. 12: Involvement of canonical Wnt signaling in 1-benzyl I3C's mechanism of action. (FIG. 12A) TOP FLASH Wnt reporter assay was performed on G361 melanoma cells treated with 1-benzyl I3C for 24 hours. Cells were transfected with either an inducible TOP luciferase reporter construct driven by tandem repeats of TCF/LEF-1 response elements (TREs) or a non-inducible TRE containing FOP construct to serve as negative control. For each condition the cells were grown in Wnt conditioned media (right panel) or conditioned media with no secreted Wnt (left panel). Subsequently the cells were treated with or without 20 μM 1-benzyl I3C and the luciferase activity was measured. The bar graph shows the results of three independent trials in triplicate±SEM (*, p<0.01). (FIG. 12B) Predictive binding simulations of 1-benzyl I3C with Wnt 5A were analyzed using the PyMol program (left side simulation). The LigPlot program examined Van der Waals interactions (within 3.5 Å) between 1-benzyl I3C and amino acids in Wnt 5A (right side simulation). (FIG. 12C) In order to validate the in-silico prediction, G361 cells were transfected with either a constitutively active LRP6-luciferase reporter construct or a construct with wild type LRP6. Subsequently the cells were treated with or without 20 μM 1-benzyl I3C for 24 hours and luciferase activity was determined. The bar graph shows the results of three independent trials in triplicate±SEM (*, p<0.01).

### Example 12: β-Catenin Mediates 1-Benzyl I3C's Regulation of MITF-M Transcript Expression and Antiproliferation

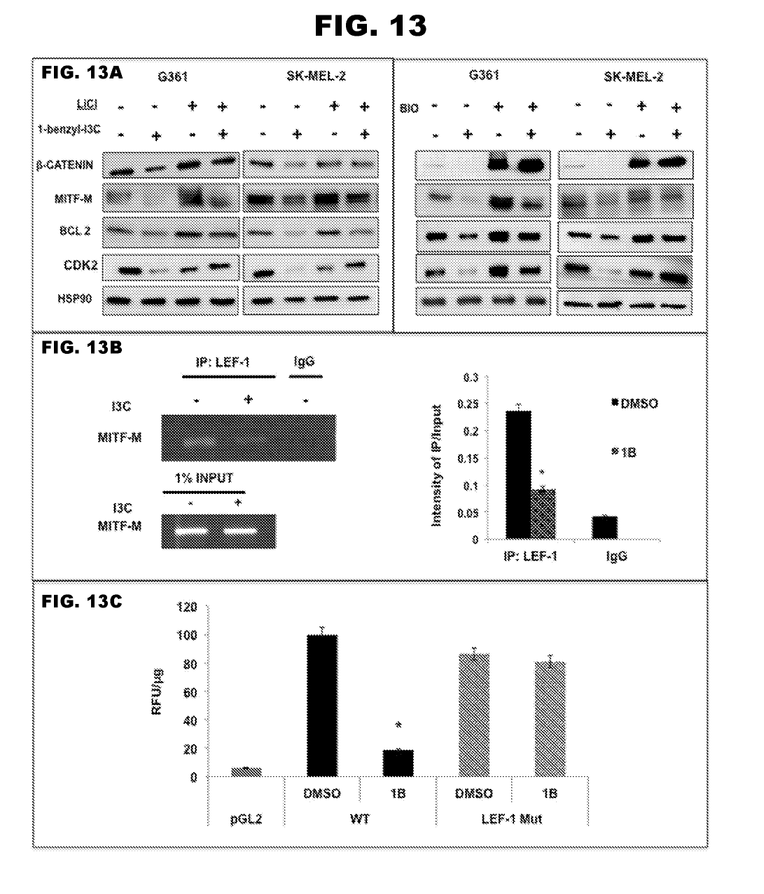

It was tested whether the MITF-M transcript inhibition by 1-benzyl I3C is a direct result to the observed downregulation of Wnt signaling. A pharmacological approach was taken to confirm the role of β-catenin in the transcriptional downregulation of MITF-M induced by 1-benzyl I3C. 1-benzyl I3C sensitive melanoma cells, G361, SK-MEL-2, were pre-treated with inhibitors of the β-catenin inhibitor GSK3β, like Lithium Chloride (LiCl) or 6-bromoindirubin-3′-oxime (BIO), which is a potent reversible ATP-competitive inhibitor of GSK3β. The treatment is continued for 3 hours prior to treatment with or without 1-benzyl I3C for 24 hours. Western blots on these cells demonstrated a significant rescue of the 1-benzyl Indole-3-carbinol induced downregulation of β-catenin, as well as its transcriptional target MITF-M and its downstream cell cycle regulator target like CDK2 and anti-apoptotic target like BC12 upon treatment with the GSK3β inhibitors (FIG. 13A). This implicated a critical role of β-catenin mediated regulation of MITF-M in 1-benzyl Indole-3-carbinol's antiproliferative effect.

β-catenin is a known transcriptional regulator of MITF-M that interacts with the co-factor TCF/LEF to bind to the putative LEF binding site on the MITF-M promoter at (−199 to −193 CTTTGAT). To further elucidate the direct outcome of downregulated β-catenin on MITF-M transcriptional activity Chromatin immunoprecipitation (ChIP) assay was performed on G361 cells treated with or without 1-benzyl I3C for 48 hours. Harvested cells were sonicated to shear the genomic DNA, which was subsequently cross-linked to protein and immunoprecipitated with either anti-LEF1 or with an IgG control antibodies. PCR analysis using primers specific to the LEF1 binding site in the MITF-M promoter, revealed that 1-benzyl I3C significantly disrupted nuclear LEF1 interactions with this promoter. One percent input was used as a loading control (FIG. 13B).

Next, to determine if the decreased binding of LEF-1 to the MITF-M promoter affects promoter activity and accounts for the observed reduction in MITF-M transcript levels, G361 melanoma cells were transiently transfected with a wild-type −333/120 MITF-M promoter-luciferase reporter plasmid (WT) and luciferase activity was determined post treatment with or without 1-benzyl I3C for 24 hours. Luciferase activity was significantly abrogated in the 1-benzyl I3C treated cells compared to the DMSO vehicle treated control, confirming the inhibitory effect of 1-benzyl I3C on the MITF promoter activity. Mutation of the LEF1 consensus site (LEF1 Mut), prevented this 1-benzyl I3C down regulation of MITF-M promoter activity, directly implicating the involvement of LEF-1 in the inhibition of MITF-M promoter activity by 1-benzyl I3C (FIG. 13C).

FIG. 13: Role of β-catenin/LEF1 mediated regulation of MITF-M expression in 1-benzyl I3C's mechanism of action. (FIG. 13A) Mutant BRAF expressing G-361 and WT BRAF expressing SK-MEL-2 cells were pre-treated with either a non-specific inhibitor—LiCl (left panel) or a specific inhibitor—BIO (right panel) of GSK3β for 3 hours. The cells were subsequently treated with or without 20 μM 1-benzyl I3C for 24 hours. Western blots were performed on whole cell extracts and probed for the indicated proteins. The results are representative of three independent experiments. (FIG. 13B) ChIP assay was performed on G361 cells treated with or without 20 μM 1-benzyl I3C for 48 hours using LEF-1 antibodies (IP: LEF-1) or the control IgG with one percent input as the loading control. The bar graphs quantify the densitometry results from three independent experiments±SEM (*, p<0.01). (FIG. 13C) Cells were transfected with reporter plasmids containing either a wild type MITF-M promoter (WT), a LEF-1 consensus site mutant (LEF-1 Mut) or PGL2 empty control vector. Luciferase specific activity was measured in cells treated with or without 20 μM 1-benzyl I3C for 24 hours. The bar graph shows the results of three independent trials in triplicate±SEM (*, p<0.01).

### Example 13: 1-Benzyl I3C and Vemurafenib Exhibit a Cooperative Antiproliferative Effect in Melanoma Cells

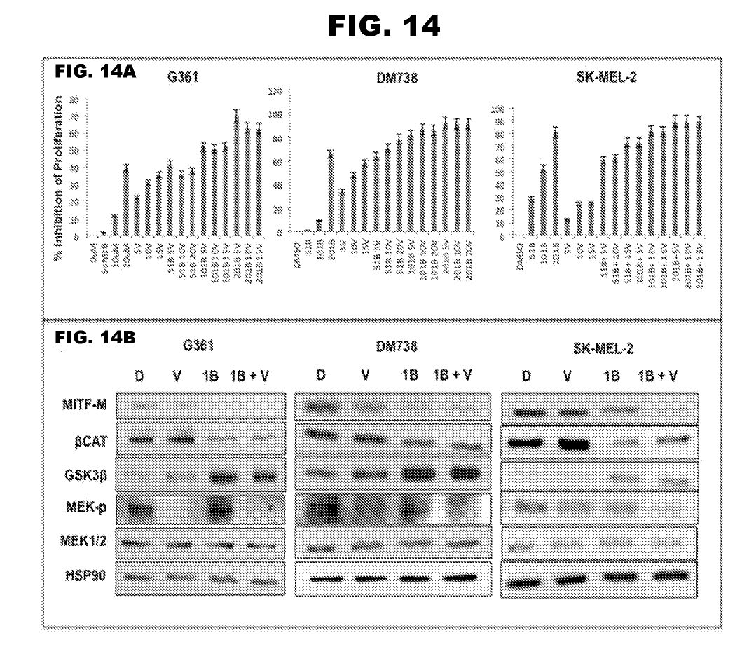

Since Vemurafenib selectively targets the oncogenic BRAF pathway and 1-benzyl Indole-3-carbinol targets the Wnt signaling pathway and both the pathways play a significant role in melanomagenesis and progression, it was hypothesized that a combination of the two compounds will synergize to cause greater antiproliferative effect than each compound alone. CCK-8 proliferation assays performed using various combinations of 1-benzyl I3C and Vemurafenib indeed showed a co-operative antiproliferative effect (FIG. 14A).

Western blots were performed to examine the effects of combinations of 20 μM 1-benzyl I3C and 10 μM Vemurafenib on MITF-M as well as the BRAF V600E and Wnt signaling was examined in G-361, DM738 and SK-MEL-2 melanoma cells, after 24 hours treatment with 1-benzyl I3C. In G361 and DM738 cells the combination of 1-benzyl I3C and Vemurafenib cooperatively down-regulated MITF-M protein levels, by inhibiting both oncogenic BRAF signaling evident from downregulated MEK-P with Vemurafenib treatment and inhibiting Wnt signaling evident from upregulated GSK3β and downregulated β-catenin protein levels with 1-benzyl I3C treatment. In SK-MEL-2 cells the inhibition on MITF-M was primarily by inhibition of Wnt signaling since the expression of wild-type BRAF rendered it not sensitive to Vemurafenib action (FIG. 14B). The combinational inhibitory effects of I3C and Vemurafenib on melanoma cell proliferation mirrored the down regulation of MITF-M levels.

FIG. 14: Effects of combinations of I3C and Vemurafenib on melanoma cell proliferation and BRAF-V600E signaling. (FIG. 14A) G-361, DM-738 and SK-MEL-2 cells were treated with the indicated combinations of 1-benzyl I3C and Vemurafenib for a suboptimal time of 24 hours and inhibition of proliferation was monitored using a CCK-8 assay. The results represent the average of three independent experiments with a mean±SEM (*, p<0.01) shown in the bar graphs. (FIG. 14B) G-361, DM-738 and SK-MEL-2 cells were treated with a combination of 20 μM I3C and/or 10 μM Vemurafenib for 24 hours and the levels of the indicated proteins determined by western blots on total cell extracts.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A pharmaceutical composition that inhibits proliferation of a cancer cell, comprising:
a) a first compound selected from indole-3-carbinol, 1-benzyl indole-3-carbinol, or a pharmaceutically acceptable salt or ester thereof; and
b) a second compound that binds to and inhibits a polypeptide selected from:
i. an oncogenic RAF polypeptide, wherein the second compound binds at a site that is distinct from the site at which the first compound binds;
ii. a polypeptide of the RAF signaling pathway selected from c-KIT, RAS, MEK, ERK, or BRN-2;
iii. a polypeptide of the Wnt-β-catenin signaling pathway; and
iv. a polypeptide of the PI3K/AKT/mTOR signaling pathway.

2. The composition of claim 1, wherein the second compound binds to and inhibits an oncogenic RAF polypeptide.

3. The composition of claim 1, wherein the second compound binds at or near the ATP pocket of the oncogenic RAF polypeptide.

4. The composition of claim 1, wherein the second compound is Vemurafenib.

5. The composition of claim 1, wherein the second compound is Dabrafenib.

6. The composition of claim 1, wherein the cancer is melanoma.

7. The composition of claim 6, wherein the cancer is BRAF-inhibitor-resistant melanoma.

8. The composition of claim 1, wherein the cancer expresses an oncogenic mutation in BRAF.

9. The composition of claim 8, wherein the cancer is colon, thyroid or lung cancer.

10. The composition of claim 1, wherein the oncogenic BRAF polypeptide comprises a BRAF-V600 mutation.

11. The composition of claim 10, wherein the BRAF-V600 mutation is V600E, V600K, V600D or V600R.

12. A method of treating cancer in a subject, the method comprising administering to the subject combined effective amounts of:
a) a first compound selected from indole-3-carbinol, 1-benzyl indole-3-carbinol, or a pharmaceutically acceptable salt or ester thereof; and
b) a second compound that binds to and inhibits a polypeptide selected from:
i. an oncogenic BRAF polypeptide, where the second compound binds at a site that is distinct from the site at which the first compound binds;
ii. a polypeptide of the RAF signaling pathway selected from c-KIT, RAS, MEK, ERK, or BRN-2;
iii. a polypeptide of the Wnt-β-catenin signaling pathway; and
iv. a polypeptide of the PI3K/AKT/mTOR signaling pathway.

13. The method of claim 12, wherein the second compound is Vemurafenib.

14. The method of claim 12, wherein the second compound is Dabrafenib.

15. The method of claim 12, wherein the cancer is melanoma.

16. The method of claim 15, wherein the cancer is BRAF-inhibitor-resistant melanoma.

17. The method of claim 12, wherein the cancer expresses an oncogenic mutation in BRAF.

18. The method of claim 17, wherein the cancer is colon, thyroid or lung cancer.

19. The method of claim any one of claims 12-18, wherein the first compound is administered orally, topically, intravenously, or intramuscularly.

20. The method of claim 12, wherein the second compound is administered orally, topically, intravenously, or intramuscularly.

21. The method of any one of claims 12-20, wherein the first compound and the second compound are administered in the same formulation.

22. The method of any one of claims 12-20, wherein the first compound and the second compound are administered in separate formulations.

23. The method of any one of claims 12-20, wherein the first compound and the second compound are administered substantially simultaneously.

24. The method of any one of claims 12-20, wherein the first compound and the second compound are administered within 1 hour to 24 hours of one another.

25. The method of any one of claims 12-20, wherein the first compound is 1-benzyl-I3C and the second compound is Vemurafenib.

26. The method of claim 25, wherein the 1-benzyl-I3C is administered in an amount of from about 1 mg/kg to about 50 mg/kg.

27. The method of claim 25, wherein the Vemurafenib is administered in an amount of from 100 mg/kg to about 500 mg/kg.


SEQUENCE LISTING (8 sequences)

SEQ ID NO: 1 - DNA, 20 bp, Artificial Sequence
  Synthetic Primer
ccgtctctcactggattggt

SEQ ID NO: 2 - DNA, 20 bp, Artificial Sequence
  Synthetic Primer
tacttggtggggttttcgag

SEQ ID NO: 3 - DNA, 19 bp, Artificial Sequence
  Synthetic Primer
tgaacgggaagctcactgg

SEQ ID NO: 4 - DNA, 20 bp, Artificial Sequence
  Synthetic Primer
tccaccaccctgttgctgta

SEQ ID NO: 5 - DNA, 35 bp, Artificial Sequence
  Synthetic Primer
gacagtgagtttgactttggcagctcgtcacttaa

SEQ ID NO: 6 - DNA, 35 bp, Artificial Sequence
  Synthetic Primer
ttaagtgacgagctgccaaagtcaaactcactgtc

SEQ ID NO: 7 - DNA, 31 bp, Artificial Sequence
  Synthetic Primer
cgtcacttaaaaaggtacctttatatttatg

SEQ ID NO: 8 - DNA, 33 bp, Artificial Sequence
  Synthetic Primer
tgttttagctagcaccaatccagtgagagacgg